(19) United States
(12) Patent Application Publication
(10) Pub. No.: US 2022/0193302 A1
(43) Pub. Date: Jun. 23, 2022

(54) MICROPOST ARRAY APPARATUS AND COMPOSITE BIOLOGICAL SCAFFOLD

(71) Applicant: Embody, Inc., Norfolk, VA (US)

(72) Inventors: Michael P. FRANCIS, Norfolk, VA (US); Kyle CHRISTENSEN, Norfolk, VA (US); Nicholas THAYER, Norfolk, VA (US)

(21) Appl. No.: 17/538,296

(22) Filed: Nov. 30, 2021

Related U.S. Application Data
(60) Provisional application No. 63/119,618, filed on Nov. 30, 2020.

(51) Int. Cl. A61L 27/24; B33Y 10/00; B33Y 30/00; B33Y 70/00; B33Y 80/00; A61L 27/36

(52) U.S. Cl. CPC A61L 27/24; B33Y 10/00; B33Y 30/00; A61L 2430/30; B33Y 80/00; A61L 27/367; B33Y 70/00

(57) ABSTRACT

A biocompatible scaffold construct may include a biocompatible hydrogel and at least one biomaterial microfiber strand wound to form a plurality of microfiber segments in proximity to one another and arranged in an organized configuration.

### CROSS-REFERENCE TO RELATED APPLICATIONS

This application claims priority to U.S. Provisional Application No. 63/119,618, filed Nov. 30, 2020, and entitled “Micropost Array Apparatus and Composite Biological Scaffold,” the entire disclosure of which is incorporated herein by reference.

### STATEMENT OF US GOVERNMENT SUPPORT

This invention was made with government support under DARPA Contract HR0011-15-90006. The US government has certain rights in the invention.

### BACKGROUND

Musculoskeletal tissue injuries are among the most common injuries treated in the United States. However, current treatment options often lead to impaired function of the injured tissue and high rates of reinjury. As such, numerous tissue engineering approaches have been developed to manufacture scaffold-like grafts aiming to facilitate the regeneration of functional native-like tissue. Recreating the biochemical, morphological, and functional properties of the targeted tissue is of particular importance.

Three-dimensional (3D) bioprinting, an additive biomanufacturing approach, is commonly implemented for the fabrication of scaffolds with potential regenerative medicine applications. 3D bioprinting enables the precise manipulation of cells and biomaterials into designed and often complex 3D geometries. However, bioprinting approaches typically utilize soft hydrogels as the primary structural material, particularly when dealing with biological polymers such as collagen [Mandrycky 2016]. The mechanical properties of these hydrogels often are orders of magnitude below those of native ligament, tendon or other tissues. As such, typical bioprinting approaches cannot adequately recreate the functional properties of musculoskeletal or other tissues, and thus are unable to produce load-bearing scaffolds for tissue repair.

Hybrid bioprinting approaches have been developed that incorporate thermoplastic polymers along with hydrogels to improve the mechanical properties of printed parts [Merceron 2015]. However, many printed synthetic materials still have limited mechanical strength and may negatively affect injury healing and tissue regeneration.

To address these challenges, numerous fiber-based tissue engineering approaches have been developed using strong, natural biomaterials such as collagen [Tamayol 2013]. These approaches build on the well-established clinical use of textiles but incorporate additional means to produce cellularized scaffolds.

Also, the use of therapeutic cells offers potential to improve the treatment of genetic, degenerative, inflammatory, and traumatic musculoskeletal disorders [O'Keefe 2019]. As opposed to biomaterial scaffolds alone, this may improve healing rates and overall regeneration and functional recovery of tissues. This may be especially true in the case of ligament- and tendon-like tissue, for which passive cellular ingrowth may be limited in the hypocellular and hypovascular environments.

Some approaches to producing biomaterial scaffolds utilize premanufactured fibers produced by traditional textile manufacturing processes of weaving, knitting, and braiding. Biomaterial fibers can be produced as feedstock for these processes by wetspinning, microfluidic spinning, biospinning, interface complexation, and melt spinning [Tamayol 2013]. Weaving can be used to create polymer scaffolds with designed porosity, morphology, and geometry by interlacing two sets of warps or wefts at right angles [Abrahamsson 2010]. Knitting is a commonly used approach for fabricating surgical meshes and forms 3D geometries from intertwining yarns or threads in a series of interconnected loops [Sahoo 2007]. Braiding is capable of forming complex biomaterial structures or patterns by intertwining multiple fiber stands [Walters 2012]. Additionally, relatively simple geometries such as bundles of parallel fibers bound by suture have been fabricated by manual assembly [Gentleman 2006].

Scaffold fabrication processes that generate biomaterial scaffolds utilizing fibers produced as an integral part of the scaffold include electrospinning, wetspinning, and direct writing. These approaches utilize processes such as solvent evaporation, polymerization within a solution bath, or temperature-based recrystallization to form microfiber scaffolds from biomaterial solutions. For example, electrospinning has been used to form randomly oriented or aligned polymer fiber mats with biomimetic surface patterns to direct tissue formation [Mauck 2009]. Wetspinning, in addition to being used to form fiber as feedstock, can be utilized to fabricate scaffolds during the fiber formation process by collecting fibers on a rotating mandrel [Kaiser 2019]. Direct writing is capable of forming fiber-based scaffolds with excellent control of porosity, fiber size, and fiber orientation [Wu 2015].

However, the post-fabrication cell seeding processes required to create cellularized scaffolds using weaving, knitting, braiding, electrospinning, wetspinning, and direct writing can be subject to human variability, and may be highly dependent on the macroscale geometry and porosity of the biomaterial scaffolds. For example, small pore sizes may limit cell infiltration during seeding, especially for scaffolds with a high thickness or complex 3D geometry. Conversely, scaffolds with high porosity may have difficulty in retaining seeded cells uniformly throughout. The dependency of cell seeding on scaffold micro- and macro-scale geometry may lead to limited control of cell distributions throughout, particularly for the fabrication of heterogeneous tissues with distinct cell populations in designed regions.

To address challenges with seeding cells onto prefabricated scaffolds, various fiber-based approaches have been developed that directly manipulate cells or cell-laden materials during the scaffold manufacturing process [Tamayol 2013]. Compared to techniques requiring cell seeding, these approaches may result in cellularized scaffolds with improved consistency and control of cell distributions throughout. Polyester threads have been coated with a cell-laden hydrogel and wrapped around a cylindrical mandrel to form 3D tubular structures [Liberski 2011]. A microfluidic system was used to form core-shell hydrogel fibers encapsulating cells which could be implanted without forming a secondary scaffold structure [Sugimoto 2011]. A micro-weaving approach was utilized to form centimeter-scale living fabrics from similar cell-laden core-shell hydrogel fibers [Onoe 2011]. However, the limited mechanical strength of cell-laden hydrogel fibers limits their ability to be processed using traditional textile fabrication approaches [Onoe 2011]. In a direct writing approach similar to typical hydrogel-based bioprinting, a cell suspension was crosslinked within a printhead to form cell-laden scaffolds from fiber-like extrusions [Ghorbanian 2014]. Overall, poor mechanical properties of scaffolds formed using these hydrogel-based approaches have limited applicability as load-bearing scaffolds for the treatment of musculoskeletal tissue injuries.

It would be desirable to address one or more of the issues discussed above.

### SUMMARY

In one aspect, the present disclosure is directed to a composite scaffold. The composite scaffold may include a biocompatible hydrogel and at least one biomaterial strand wound to form a plurality of segments in proximity to one another and arranged in an organized configuration.

In another aspect, the present disclosure is directed to an apparatus for making a composite scaffold. The apparatus may include a first array of microposts and a second array of microposts arranged spaced from the first array of microposts. The microposts may be configured to receive a microfiber strand to form a plurality of segments, wherein at least some of the plurality of segments are arranged in a substantially aligned configuration.

In another aspect, the present disclosure is directed to a method of making a composite scaffold. The method may include dispensing a microfiber strand in a biocompatible hydrogel sheath and winding the microfiber strand around a plurality of microposts to form a plurality of segments ensheathed by a biocompatible hydrogel and arranged in an organized configuration.

Other systems, methods, features, and advantages of the embodiments will be, or will become, apparent to one of ordinary skill in the art upon examination of the following figures and detailed description. It is intended that all such additional systems, methods, features, and advantages be included within this description and this summary, be within the scope of the embodiments, and be protected by the following claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The embodiments can be better understood with reference to the following drawings and description. The components in the figures are not necessarily to scale, with emphasis instead being placed upon illustrating the principles of the embodiments. Moreover, in the figures, like reference numerals designate corresponding parts throughout the different views.

FIG. 1 is a schematic view of a portion of an apparatus for making a composite scaffold according to an exemplary embodiment;

FIG. 2 is a schematic perspective view of a rack including opposing arrays of microposts configured to receive collagen microfibers;

FIG. 3 is a schematic view of a collagen microfiber strand wound in an organized fashion between two opposing arrays of microposts;

FIG. 4 is a schematic view of a portion of a 3D printing device and a receiving assembly;

FIG. 5 is a schematic enlarged view of a coaxial needle tip with a flexible extension fitted to the outer conduit;

FIG. 6 is a schematic illustration of a print head path for winding a collagen microfiber strand around the microposts;

FIG. 7 is a schematic enlarged view of a portion of a composite scaffold formed using the materials and methods described herein;

FIG. 8 is a flowchart illustrating steps of a method of making a composite scaffold according to an exemplary embodiment;

FIG. 9 is a macroscopic image of a printed scaffold submerged in media in a microwell plate;

FIG. 10 is a macroscopic image of a printed scaffold held by forceps;

FIG. 11 is a transmitted light microscopic image of an acellular printed scaffold including a pattern of parallel collagen microfiber segments;

FIG. 12 is a transmitted light microscopic image of a cellular printed scaffold including a pattern of parallel collagen microfiber segments;

FIG. 13 is a microscopic image of a pattern formed by wrapping collagen fiber around a micropost array after two days of culture;

FIG. 14 is the microscopic image from FIG. 13 at twice the magnification of FIG. 13, showing visibly extended cell morphology indicating interaction with the hydrogel environment;

FIG. 15A is a fluorescence image of a scaffold after seven days of culture, showing cytoskeleton protein vimentin, cell nuclei using DAPI, and collagen fiber autofluorescence at 495 nm;

FIG. 15B is a composite image from 12 fields of view of a printed cellular construct after 7 days of culture;

FIG. 16 is a graph showing the results of an alamarBlue assay indicating metabolic activity for scaffolds printed with human tenocytes after 7 and 14 days of culture;

FIG. 17 is a graph showing peak load of acellular and cellular composite collagen microfiber scaffolds printed using the micropost array approach after seven days of constant-tension bioreactor culture;

FIG. 18 is a graph showing ultimate tensile strength (UTS) of acellular and cellular composite collagen microfiber scaffolds printed using the micropost array approach after seven days of constant-tension bioreactor culture;

FIG. 19 is a graph showing Young's modulus of acellular and cellular composite collagen microfiber scaffolds printed using the micropost array approach after seven days of constant-tension bioreactor culture;

FIG. 20 is a graph showing cross-sectional area of acellular and cellular composite collagen microfiber scaffolds printed using the micropost array approach after seven days of constant-tension bioreactor culture;

FIG. 21 is a graph showing strain at break of acellular and cellular composite collagen microfiber scaffolds printed using the micropost array approach after seven days of constant-tension bioreactor culture;

FIG. 22A is an image showing the creation of a volumetric muscle loss (VML) injury in a rodent test specimen;

FIG. 22B is an image showing initial placement of an implant in the VML injury created in the rodent;

FIG. 22C is an image showing corner attachment points at which the implant is secured to the native tissue of the rodent;

FIG. 22D is an image showing the injury site with a tissue flap (“fascia”) sutured over the implant as placed in FIGS. 49B and 49C;

FIG. 22E is a graph illustrating the body weight of the animals during the weeks following the implantation of the implant as shown in FIGS. 49B-D;

FIG. 22F is a graph illustrating the weight of the defects (i.e., how much tissue was removed) for each animal at the time of surgery;

FIG. 22G is a graph illustrating the baseline torque generated by each animal prior to defect creation;

FIG. 22H is a graph illustrating the torque generated by each animal at 4, 8, and 12 weeks post-repair;

FIG. 22I is a graph illustrating the post-repair torque generation as a percentage of baseline torque;

FIG. 23A is an H&E photomicrograph showing a histological assessment of the tibialis anterior (TA) muscle of a control rodent with no injury;

FIG. 23B is an H&E photomicrograph showing a histological assessment of the tibialis anterior (TA) muscle of a rodent with a VML injury to which no repair was made;

FIG. 23C is an H&E photomicrograph showing a histological assessment of the tibialis anterior (TA) muscle of a rodent with a VML injury which has been repaired with an acellular implant;

FIG. 23D is an H&E photomicrograph showing a histological assessment of the tibialis anterior (TA) muscle of a rodent with a VML injury which has been repaired with a cellular implant;

FIG. 23E is an H&E photomicrograph of an acellular implant implanted in a rodent specimen;

FIG. 23F is an H&E photomicrograph of a cellular implant implanted in a rodent specimen;

FIG. 24 is a collection of H&E photomicrographs showing prominent new muscle fibers (labeled with “MF”) growing within the scaffold construct implants as demarcated by the implant collagen fibers (labeled with “*”);

FIG. 25 is a collection of Masson's Trichrome photomicrographs showing the presence of collagen within and around the implant region and defect area from each identified group;

FIG. 26A is photomicrograph of a TA muscle belly processed for analysis using SMASH (a semi-automated muscle fiber analysis software) showing an uninjured control;

FIG. 26B is photomicrograph of a TA muscle belly processed for analysis using SMASH showing a specimen with VML injury with no repair;

FIG. 26C is photomicrograph of a TA muscle belly processed for analysis using SMASH showing a specimen with VML injury that has been repaired with an acellular implant;

FIG. 26D is photomicrograph of a TA muscle belly processed for analysis using SMASH showing a specimen with VML injury that has been repaired with a cellular implant;

FIG. 26E is a colorized output from the SMASH software identifying individual muscle fibers within sections corresponding to FIG. 26A;

FIG. 26F is a colorized output from the SMASH software identifying individual muscle fibers within sections corresponding to FIG. 26B;

FIG. 26G is a colorized output from the SMASH software identifying individual muscle fibers within sections corresponding to FIG. 26C;

FIG. 26H is a colorized output from the SMASH software identifying individual muscle fibers within sections corresponding to FIG. 26D;

FIG. 26I is a graph showing the total fiber count of the four experimental groups (uninured control (Ctrl), no repair (NR), acellular implant (AI), and cellular implant (CI));

FIG. 26J is a graph showing median fiber cross-sectional area (FCSA) of the four experimental groups; and

FIG. 26K is a graph showing the product of fiber count and FCSA for the four experimental groups.

### DETAILED DESCRIPTION

The novel biomanufacturing approaches described in this specification provide improved methods to fabricate cellularized scaffolds for regenerative medicine applications. Scaffolds are fabricated from strong, stable microfibers of clinical-grade collagen with biochemical and mechanical properties similar to those of native tissue. This fiber is uniformly and controllably coated with cells during scaffold fabrication. Any cell type may be chosen based on intended application and may include stem cells, tenocytes, chondrocytes, myoblasts, osteoblasts, or numerous tissue-specific cell types. Appropriate cell culture media and material additives should be utilized to facilitate survival of chosen cell types. Scaffolds preferably are formed with microstructural cues to signal cell alignment as well as with a designed porosity, fiber patterns, and macroscopic dimensions appropriate for their intended use. The fabrication process described herein is rapid, repeatable, scalable, and may be automated. The scaffolds, as described herein, mimic the biological, morphological, and functional properties of native musculoskeletal tissues.

The embodiments described herein are related to a composite scaffold, methods for making the composite scaffold, and an apparatus for making the composite scaffold. Such scaffolds include a plurality of aligned collagen microfiber segments embedded in a biocompatible hydrogel. Such scaffolds may be suitable for use as tendon or ligament grafts or to support other biomechanical surgical repairs.

To assist and clarify the subsequent description of various embodiments, various terms are defined herein. Unless otherwise indicated, the following definitions apply throughout this specification (including the claims). For consistency and convenience, directional adjectives are employed throughout this detailed description corresponding to the illustrated embodiments.

The term “biomaterial,” as used throughout this detailed description and in the claims, refers to naturally-derived protein, glyocoprotein, and glycosaminoglycan-based biopolymers and their synthetic counterparts. Possible biomaterials include those which have been utilized across 3D bioprinting approaches including several fiber-based biomanufacturing approaches in particular. These may include, but are not limited to, collagen, elastin, fibronectin, fibrinogen, silk, synthetic polymers, proteoglycans and hyaluronic acid. [See, Skardal 2014 and Tamayol 2013.]

With respect to collagen, contemplated types include atelocollagen, telocollagen, gelatin, and may be collagen sources, such as recombinant human collagen, porcine collagen, bovine collagen, jellyfish collagen, and mixtures thereof. A person skilled in the art will understand that fibers will be produced having tensile strengths, resiliency, elasticity and toughness appropriate for the particular functions and uses of a given implant as discussed herein.

The term “fiber,” as used throughout this detailed description and in the claims, refers to fiber, thread, or filament having a high ratio of length to diameter and normally used as a unit. The term “microfiber” is used synonymously due to the size scale of the fiber used in preferred embodiments. The term “strand” refers to an individual item of fiber, whereas a fiber-based construct may consist of many individual fiber strands. The term “segment” refers to a length of fiber in reference to a spatial location, such as a fiber segment existing at a specific location of a fiber-based construct. Throughout, the above terms may include fiber comprised of multiple sub-fibers assembled by secondary or tertiary assembly processes such as braiding.

The term “lateral,” as used throughout this detailed description and in the claims, refers to a side-to-side direction extending along the width of a component.

The term “longitudinal,” as used throughout this detailed description and in the claims, refers to a direction extending along the length of a component.

The term “micropost,” as used throughout this detailed description and in the claims, refers to an anchoring point, protrusion, or structure onto or around which fiber may be wrapped or otherwise removably attached. A person skilled in the art will understand that microposts shown in the preferred embodiment are one kind of anchoring structure among many possible anchoring structure geometries.

The term “vertical,” as used throughout this detailed description and in the claims, refers to a direction generally perpendicular to both the lateral and longitudinal directions.

The term “scaffold,” as used throughout this detailed description and in the claims, refers to a 2D or 3D assemblage of fiber. The terms “construct” and “macrostructure” are used synonymously. The term “graft” refers to a scaffold which is intended for implantation as a medical device as used in preferred embodiments.

It will be understood that each of these directional adjectives may be applied to individual components of a discussed device or apparatus. The term “upward” refers to the vertical direction heading away from a ground surface, while the term “downward” refers to the vertical direction heading toward the ground surface. Similarly, the terms “top,” “upper,” and other similar terms refer to the portion of an object substantially furthest from the ground in a vertical direction, and the terms “bottom,” “lower,” and other similar terms refer to the portion of an object substantially closest to the ground in a vertical direction.

For purposes of this disclosure, the term “fixedly attached” shall refer to two components joined in a manner such that the components may not be readily separated (for example, without destroying one or both of the components). Exemplary modalities of fixed attachment may include joining with permanent adhesive, rivets, stitches, nails, staples, welding or other thermal bonding, or other joining techniques. In addition, two components may be “fixedly attached” by virtue of being integrally formed, for example, in a molding process.

For purposes of this disclosure, the term “removably attached” shall refer to the joining of two components in a manner such that the two components are secured together, but may be readily detached from one another. Examples of removable attachment mechanisms may include hook and loop fasteners, friction fit connections, interference fit connections, threaded connectors, cam-locking connectors, and other such readily detachable connectors.

The term “strand” includes a single fiber, filament, or monofilament, as well as an ordered assemblage of fibers having a high ratio of length to diameter and normally used as a unit.

The present disclosure is directed to a composite scaffold including a plurality of substantially aligned collagen microfiber segments embedded in a biocompatible hydrogel. The present disclosure is further directed to a method and apparatus for making such a composite scaffold. It will be noted that the disclosed system and process may be applicable to other types of fibers besides collagen microfibers, including other natural fibers and/or synthetic materials, as well as to pliable polymers such as suture, or soft steel wire, for example, and other products that a person skilled in the art will understand to be appropriate for use in preparing similar scaffolds.

The scaffold is generally formed by dispensing a collagen microfiber strand in such a manner that the dispensed strand is coated in a biocompatible hydrogel as a cellular binder. For purposes of the present invention, the microfiber may be dispensed by drawing a microfiber strand under tension or by extruding it. The collagen microfiber strand is dispensed from a center lumen of an inner conduit of a coaxial needle, with the biocompatible hydrogel precursor being dispensed from an annular lumen around the inner conduit. The hydrogel precursor is dispensed from the annular lumen via controlled actuation of a plunger of a syringe containing hydrogel precursor solution. In a preferred embodiment, the microfiber is provided from a spool. The extrusion of an exemplary collagen fiber is disclosed by Francis et al., U.S. Patent Application Publ. No. 2020/0246505, published on Aug. 6, 2020, and entitled “Microfluidic Extrusion,” which describes a product having ultimate tensile strength, modulus of elasticity, and strain at break comparable to those of native human tendons and ligaments. The entire disclosure of U.S. Patent Application Publ. No. 2020/0246505 is incorporated herein by reference.

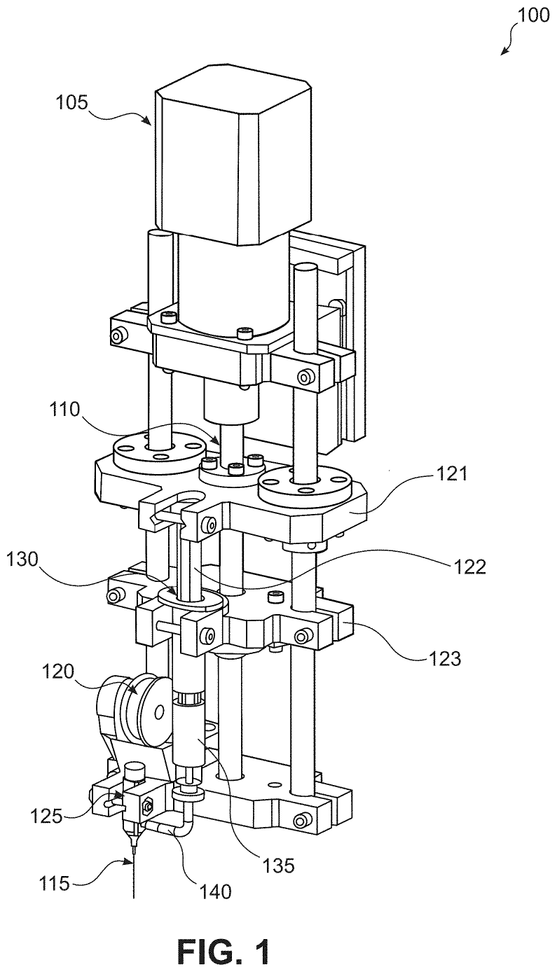

FIG. 1 is a schematic view of a portion of an apparatus for making a composite scaffold according to an exemplary embodiment. As shown in FIG. 1, a printing device 100 may include a 3D printing assembly further including features configured for dispensing of collagen microfiber sheathed in a biocompatible hydrogel. The 3D printing assembly is provided in order to control the motion of printing device 100 in the X, Y, and Z axes. It will be understood that, in some embodiments, the printing device may have 4, 5, or 6 axes as well as having additional degrees of freedom for the printhead and receiving substrate. Further, it will also be understood that the printing device may be configured to produce two-dimensional (2D) or three-dimensional (3D) constructs. In addition, in some embodiments, the system may be configured to produce cellular scaffolds, thus adding a fourth dimension to the constructs.

It will also be understood that the constructs may be produced with a variety of geometries. For example, scaffolds may be produced having planar sheet-like geometries, prismatic geometries, rounded or cylindrical geometries, and other complex 3D geometries based on CAD models. The biomaterial strands forming these macroscopic geometries may be aligned substantially parallel to one another or be partially aligned or substantially nonaligned, with strand orientation and spacing varying in three dimensions. In addition, some constructs may have both aligned and non-aligned microfiber segments.

As shown in FIG. 1, printing device 100 may include a stepper motor 105 and a lead screw 110 configured to be driven by stepper motor 105. Lead screw 110 may be rotated in order to move a first platform 121 relative to a second platform 123 in order to actuate a plunger 122 of a syringe 130, which may contain a biocompatible hydrogel precursor solution 135. Upon rotation of screw 110, hydrogel solution 135 may be pushed through a conduit 140 into and through an annular conduit of a coaxial needle 125.

At the same time hydrogel precursor solution 135 is dispensed, a collagen microfiber strand 115 may be dispensed through an inner conduit of coaxial needle 125. It will be noted that the microfiber strand may be premanufactured and used as a feedstock. Accordingly, as shown in FIG. 1, a spool 120 may contain collagen microfiber strand 115. In order to effectuate the dispensation, a free end of collagen microfiber strand 115 may be fixed (e.g., tied) to a frame or micropost or other anchoring structure. Then, as the print head is moved, collagen microfiber strand 115 is pulled off of spool 120 and through coaxial needle 125. As this happens, collagen microfiber strand 115 is coated with hydrogel precursor solution 135.

In order to organize the collagen microfiber as it is pulled off the spool and fed through the coaxial needle, a rack may be provided with a plurality of microposts, and the collagen microfiber strand may be wound around these microposts in an organized configuration.

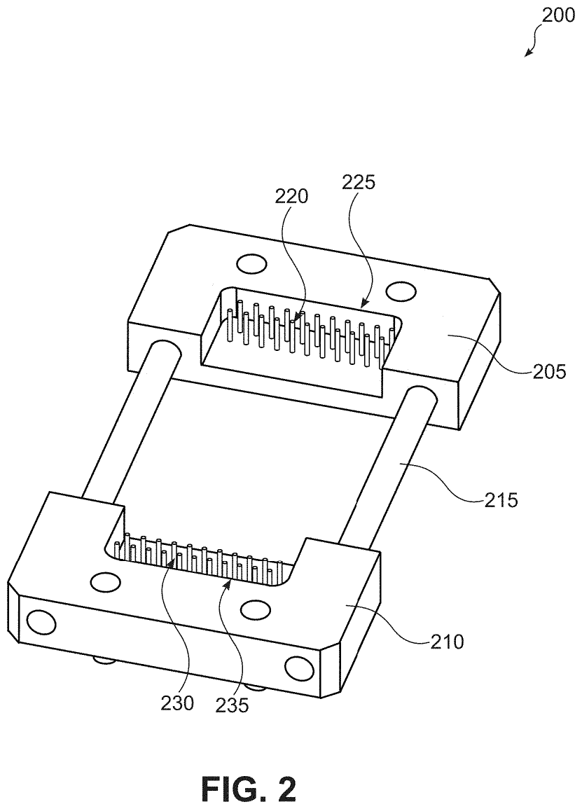

FIG. 2 is a schematic perspective view of a rack including opposing arrays of microposts configured to receive collagen microfibers. As shown in FIG. 2, an apparatus for making a composite scaffold may include a rack 200 configured for receiving collagen microfiber. In some embodiments, rack 200 may include a first portion 205 and a second portion 210 connected by rails 215. In some embodiments, first portion 205 and second portion 210 may be slidably movable with respect to one another along rails 215. This sliding ability may facilitate removal of a scaffold once the scaffold is built.

As shown in FIG. 2, rack 200 may further include a first array 220 of microposts. In some embodiments, first array 220 may be disposed in a recess 225 of first portion 205 of rack 200. In addition, rack 200 may include a second array 230 of microposts arranged spaced from and opposite first array 220 of microposts. For example, as shown in FIG. 2, second array 230 may be disposed in a second recess 235 of second portion 210 of rack 200.

The microposts are configured to receive a continuous microfiber strand that forms a plurality of adjacent microfiber segments that are arranged in a substantially aligned configuration.

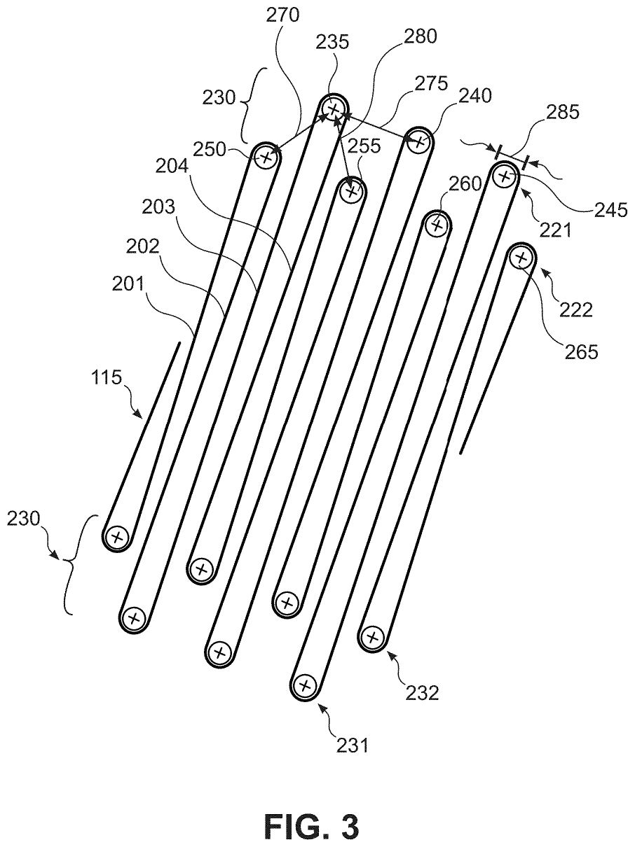

FIG. 3 is a schematic illustration of a collagen microfiber strand wound in an organized fashion between two opposing arrays of microposts. In some embodiments, the microposts in first array 220 and second array 230 may be staggered. For example, as shown in FIG. 3, first array 220 may include a first row 221 of microposts and a second row 222 of microposts offset from first row 221. Similarly, second array 230 may include a third row 231 of microposts and a fourth row 232 of microposts offset from first row 221.

As shown in FIG. 3, collagen microfiber strand 115 may be wound back and forth between the microposts of first array 220 and second array 230 in an organized configuration. Further, as shown in FIG. 3, the wound strand may form a plurality of substantially aligned microfiber segments. For example, strand 115 may be wound to form a first microfiber segment 201, a second microfiber segment 202, a third microfiber segment 203, a fourth microfiber segment 204, and so on. In some embodiments, the microfiber segments may be substantially parallel to one another, as shown in FIG. 3. In other embodiments, the micropost arrays and/or the winding of the microfibers may be such that the microfiber segments may be disposed at oblique angles to one another or be arranged in other geometric configurations.

The collagen microfiber strand and microposts may be any suitable sizes. In addition, the spacing between microposts may also vary. The following dimensions are exemplary only and it will be understood that variations may be made to such dimensions within the scope of the disclosed concepts. It will also be understood that the number of microposts used and the spacing therebetween may be selected in order to provide a composite scaffold of the desired width. Similarly, the spacing between micropost arrays may be selected to provide a composite scaffold of a desired length.

As shown in FIG. 3, first row 221 may include a first micropost 235, a second micropost 240, and a third micropost 245. Second row 222 may include a fourth micropost 250, a fifth micropost 255, a sixth micropost 260, and a seventh micropost 265. It will be noted that the number of microposts in each array is reduced in FIG. 3 for purposes of clarity. Reference can be made to FIG. 2 for an illustration of a rack having many more microposts (specifically 21 microposts in each illustrated array). A person skilled in the art will understand that the number of microposts may be larger than the specific embodiments disclosed herein and that the number of microposts should be determined with a particular product or use in mind.

In some embodiments, the collagen microfiber strand may be ribbon-shaped, with a width of approximately 50 micrometers and a thickness of approximately 5 micrometers. For such a strand, microposts with a diameter of approximately 200 micrometers may be used. For example, as shown in FIG. 3, third micropost 245 may have a diameter 285. In some embodiments, diameter 285 may be approximately 200 micrometers.

In addition, interpost spacing within each array may be approximately 1 mm. FIG. 3 illustrates a first distance 270 between first micropost 235 and fourth micropost 250; a second distance 275 between first micropost 235 and second micropost 240; and a third distance 280 between first micropost 235 and fifth micropost 255. Each of first distance 270, second distance 275, and third distance 280 may be approximately 1 mm. Accordingly, all microposts surrounding first micropost 235 may be substantially equally spaced from first micropost 235, for example at distances of approximately 1 mm.

The collagen microfiber strand may be wound about the microposts in a fluid bath that reacts with the hydrogel precursor fluid that ensheathes the microfiber strand and causes the hydrogel precursor to solidify into a hydrogel. That is, the microfiber strand ensheathed in the hydrogel precursor is dispensed into a bath of another solution that facilitates a chemical, physical, or other reaction resulting in the hydrogel precursor being converted to a hydrogel. Accordingly, this reaction between the hydrogel precursor and the fluid bath allows one to start with a liquid cell suspension and end up with a stable solid hydrogel.

In some embodiments, the fluid bath may include a crosslinking solution, such as a thrombin solution. In other embodiments, exemplary hydrogel precursor/fluid bath combinations may include fibrinogen+thrombin, fibrinogen/thrombin+Factor XIII, alginate+ionic compound, collagen+enzymatic solution, silk+enzymatic solution, or gelatin+enzymatic solution.

In addition to reacting with the hydrogel precursor to produce a hydrogel, the fluid bath also provides a hydrated physiological environment in order to maintain cell health. Accordingly, the fluid bath may provide a physiological environment having suitable temperature, pH, hydration, and biological compounds to support and maintain cell viability and health.

In order to provide this physiological environment, the manufacturing apparatus also may include a reservoir configured to receive the rack of micropost arrays and to submerge the rack in an appropriate fluid solution. This reservoir may be disposed in a fixture that can be manually moved in translation and rotation with respect to the printing device.

To maintain sterility, the entire physical system may be located within a biosafety cabinet or filtered laminar flow hood, and all components may be handled aseptically.

As discussed above, the collagen microfiber strand is dispensed coaxially within a sheath of biocompatible hydrogel precursor solution. An example of such hydrogel precursor solution is a fibrinogen solution. When the fibrinogen solution is dispensed into the thrombin crosslinking solution bath during printing, the fibrinogen rapidly solidifies to form a stable biocompatible fibrin gel. In some embodiments, cells may be suspended within the hydrogel precursor solution. Accordingly, the fibrinogen may be, but is not necessarily, a cell suspension.

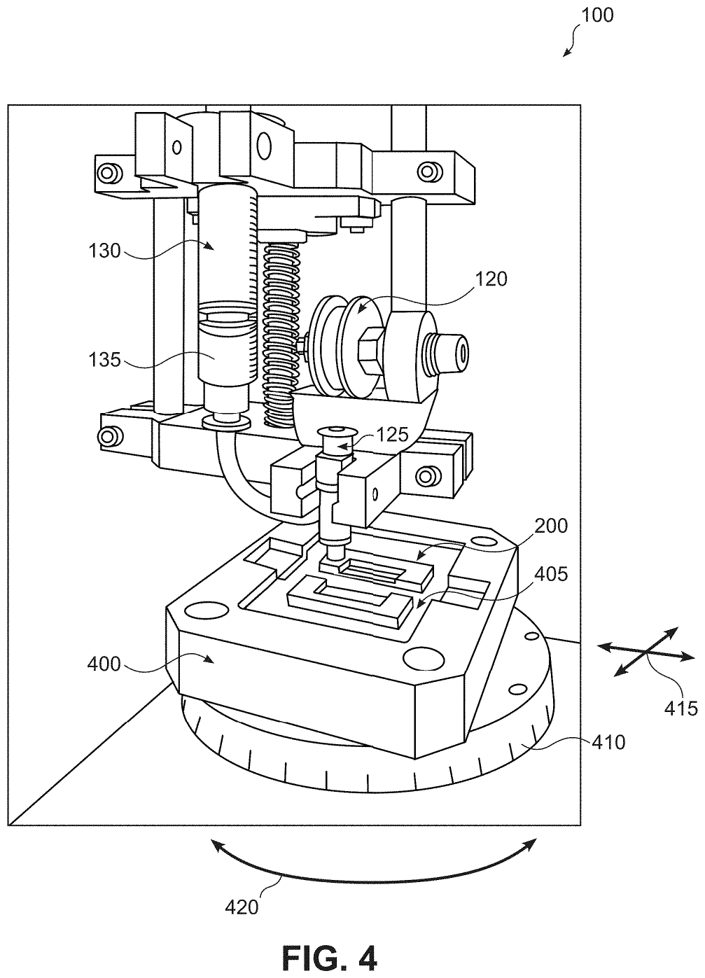

FIG. 4 is a schematic view of a portion of a 3D printing device and a receiving assembly. As shown in FIG. 4, a fixture 400 may include a reservoir 405 configured to contain rack 200 submerged in a crosslinking solution.

As further shown in FIG. 4, fixture 400 may be mounted on a stage 410 which is moveable in translation with respect to printing device 100, which is illustrated by a pair of arrows 415. In addition, stage 410 may enable fixture 400 to be rotated with respect to printing device 100, as illustrated by an arrow 420. This mobility of fixture 400 may facilitate setup of the manufacturing process. That is, stage 410 is utilized to ensure that the position and directional orientation of micropost array 200 is precisely known with respect to the position and directions of motion of printing device 100. For example, stage 410 is used to ensure that the zero point or “home location” of printing device 100 is precisely known with respect to micropost array 200. Thus, in use, the 3D printing assembly moves printing device 100 to the “home location”, then stage 410 is translated to ensure that coaxial needle 125 is positioned precisely between microposts within micropost array 200 before printing. In addition, stage 410 is also used to ensure that the lateral and longitudinal directions of motion of printing device 100 are precisely aligned with the lateral and longitudinal directions of micropost array 200. For example, the 3D printing assembly moves printing device 100 first in a purely longitudinal direction, then in a purely lateral direction. After each motion of printing device 100, stage 410 is rotated to ensure that the coaxial needle 125 is positioned precisely between microposts within micropost array 200 before printing. Once the microfiber strand is fixed to a micropost, subsequent movement of printing device 100 will result in collagen microfiber strand being drawn through the needle off of spool 120.

In some embodiments, the coaxial needle may be fitted with a flexible extension on the outer conduit in order to protect the collagen microfiber strand from damage. During dispensing of the strand, it is pulled from the tip of the inner conduit of the coaxial needle at nearly a 90-degree angle with respect to the central axis of the needle. Accordingly, in order to protect the strand from damage that may occur from pulling the strand across a relatively sharp end of the needle, a flexible extension may be included at the end of the outer conduit of the coaxial needle. Because the flexible extension bends with respect to the central axis of the needle, the strand comes out of the flexible extension at less than a 90 degree angle. In addition, the tip of the flexible extension may be much softer than a metal needle tip. The reduced angle and soft extension tip may both contribute to reducing possible damage to the strand when being drawn out of the needle.

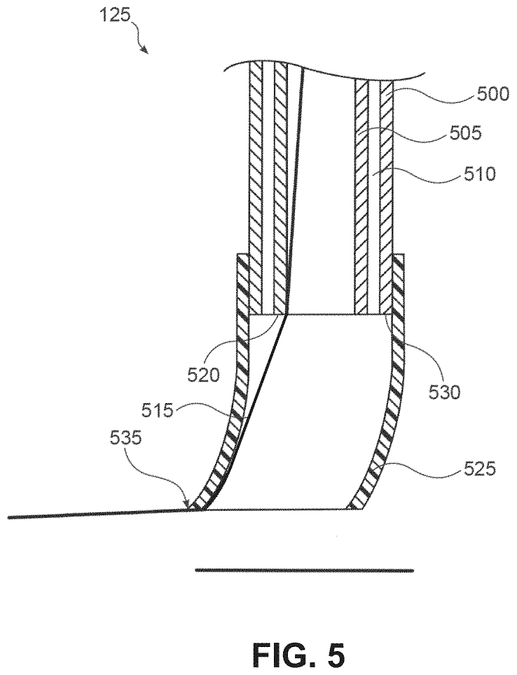

FIG. 5 is a schematic enlarged cross-sectional view of a coaxial needle tip with a flexible extension fitted to the outer conduit. As shown in FIG. 5, coaxial needle 125 may have an outer conduit 500 and an inner conduit 505. Outer conduit 500 and inner conduit 505 may be formed of a relatively rigid material, such as stainless steel or rigid plastic. An annular outer lumen 510 is defined between outer conduit 500 and inner conduit 505. Annular outer lumen 510 may be configured to dispense the hydrogel precursor sheath about the collagen microfiber strand 515 as it is drawn through inner conduit 505.

In order to protect the strand from damage when being drawn through the tip 520 of inner conduit 505, a flexible extension 525 (for example, formed of rubber, silicone, flexible plastic, etc.) may be fitted and may extend from a tip 530 of outer conduit 500. Accordingly, as shown in FIG. 5, a tip 535 of flexible extension 525 may flex with the direction strand 515 is being pulled out of needle 125.

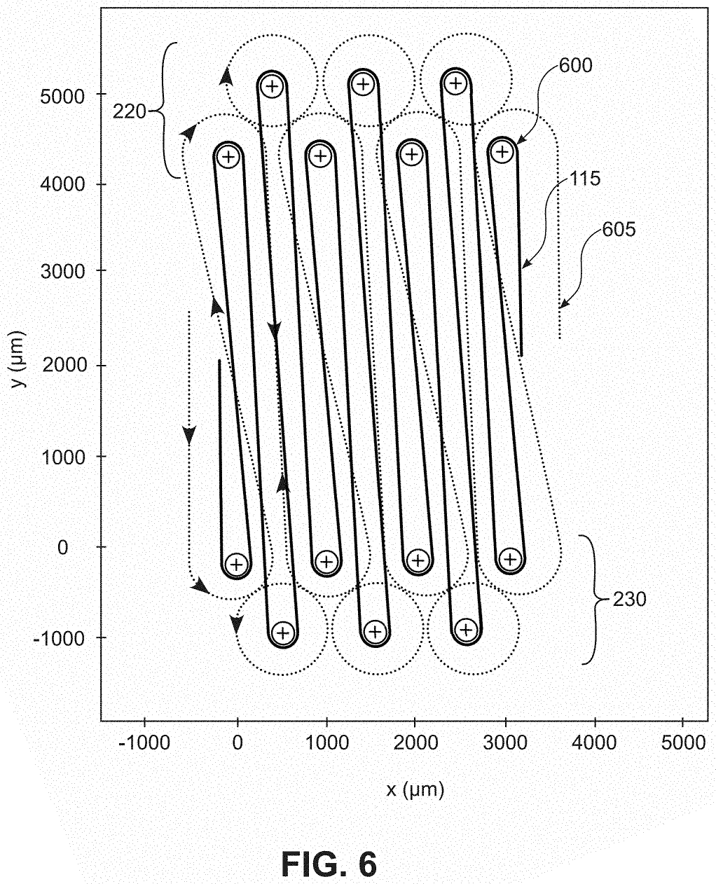

FIG. 6 is a schematic illustration of a print head path for winding a collagen microfiber strand around the microposts. As shown in FIG. 6, collagen microfiber strand 115 may be wrapped around microposts 600 by a print head that travels along the dotted path 605. That is, the coaxial needle tip may be routed along the dotted path 605 shown in FIG. 6. It will be noted that, path 605 doubles back on itself in several areas. In addition, path 605 generally remains equally spaced from all microposts 600. For example, in the embodiment shown in FIG. 6, microposts 600 are spaced approximately 1 mm (i.e., about 1000 micrometers) from one another. Accordingly, path 605 may remain at least 0.5 mm away from each micropost 600. It will also be noted that multiple strands may be wound around the microposts, thus forming multiple layers of microfibers. In some embodiments, the same strand may be continuously wound through microposts 600 to form layer upon layer of microfibers in differing vertical planes and in a determined thickness. Generally, the thickness of each layer may be approximately equal to the diameter of the fiber forming the layer. The orientation of fiber (parallel, orthogonal, or angled) may vary within in each layer or between layers. A person skilled in the art will understand that fiber orientation can be chosen and controlled throughout a fabricated structure to modulate its stability and mechanical properties in varying directions.

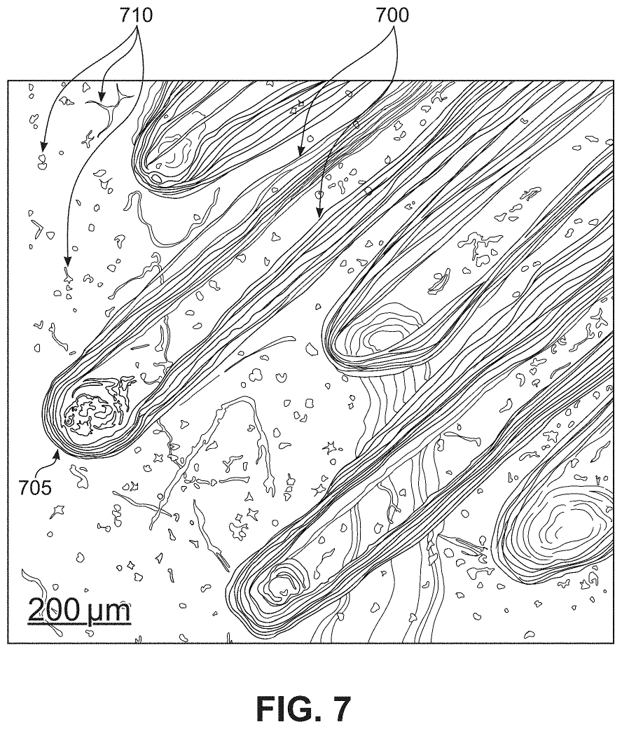

FIG. 7 is a schematic enlarged view of a portion of a composite scaffold formed using the materials and methods described herein. As shown in FIG. 7, the scaffold may include a collagen microfiber strand that may be arranged in a plurality of organized microfiber segments 700. As shown in FIG. 7, microfiber segments 700 may be substantially aligned with one another. In some embodiments, microfiber segments 700 may be substantially parallel to one another. In some embodiments, non-aligned/non-parallel microfiber segments may be utilized. In addition, it will be noted that multiple layers of microfibers 700 are stacked on top of one another.

Microfiber segments 700 may include loops 705 from where they were wrapped around the microposts during production. In addition, FIG. 7 also shows a plurality of cells 710 disposed in a cellular biocompatible hydrogel in which microfiber segments 700 are disposed. In some embodiments, the hydrogel may be crosslinked as part of the manufacturing process, for example, by the crosslinking solution in which the scaffold is produced.

FIG. 7 illustrates an approximate scale, which indicates that the microfiber segments are approximately 200 micrometers apart from one another, and that the loops 705 have a diameter of approximately 200 micrometers, which is produced by microposts having a diameter of approximately 200 micrometers. It will be noted that, in some embodiments, the segments may have a different spacing between them, that is, greater or less than about 200 micrometers. A person skilled in the art will understand that fiber spacing is dependent on fiber diameter and micropost array dimensions, and may be chosen based on the desired total amount of fiber and macroscopic dimensions of produced constructs.

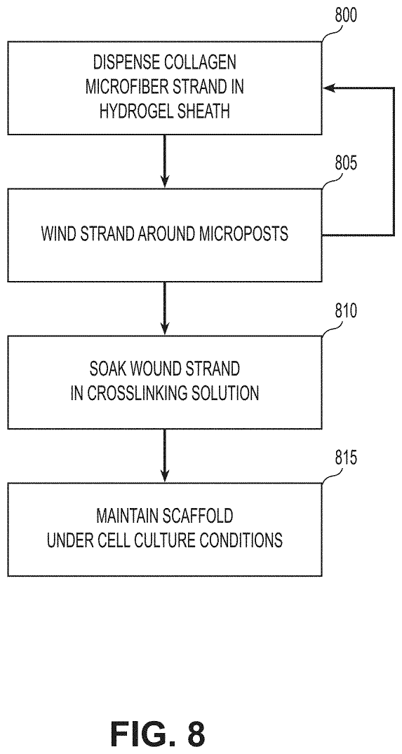

FIG. 8 is a flowchart illustrating steps of a method of making a composite scaffold according to an exemplary embodiment. As shown in FIG. 8, the method includes dispensing a collagen microfiber strand in a biocompatible hydrogel precursor sheath. (Step 800.) In addition, at step 805, the method may include winding the collagen microfiber strand around a plurality of microposts to form a plurality of substantially aligned collagen microfiber segments embedded in biocompatible hydrogel. As discussed above, winding the collagen microfiber strand around the plurality of microposts may form a plurality of substantially parallel microfiber segments. Steps 800 and 805 may be repeated to form multiple layers of the microfibers.

Further, at step 810, the method may include soaking the wound strand in a crosslinking solution. For example, as discussed above, winding the collagen microfiber strand around the microposts may be performed in a reservoir containing a bath of crosslinking solution. That is, the soaking is performed simultaneously with the winding of the strand about the microposts.

In addition, since the scaffold is a biological tissue, it should be maintained in suitable conditions. After printing is complete, the scaffold may be removed from the micropost array and placed in a cell culture vessel such as a dish or multi-well plate. For example, at step 815, the method further includes maintaining the microfiber and hydrogel scaffold under typical cell culture conditions. In some cases, the cell culture conditions may be sustained for as many as seven or more days without significant degradation of strength or microfiber organization.

The following is a further description of the materials and methods used to produce the scaffolds discussed herein.

A novel additive manufacturing approach was developed to produce cellularized composite scaffolds consisting of a biocompatible hydrogel reinforced by highly aligned, strong collagen microfiber. Specifically, in a preferred embodiment, a robotic printhead wraps collagen microfiber in-between and around an array of microposts while extruding or dispensing a cell-laden hydrogel precursor. The array of microposts are disposed within a crosslinking solution bath, which acts to solidify the cell-laden hydrogel as it is extruded or dispensed around the simultaneously drawn collagen fiber. This results in composite three-dimensional (3D) structures consisting of layers of parallel fibers of designed patterns and dimensions, surrounded by cell-laden hydrogel which maintains the macroscopic shape and orientation of the fiber. Millimeter- or centimeter-scale structures are printed with designed patterns and dimensions from collagen fiber with a width on the order of about 50 μm and thickness around 5 μm (ribbon shaped) using microposts with a diameter of about 200 μm and inter-post spacing within the array of about 1 mm.

To enable this technology, a custom extrusion printhead (see FIG. 1) was designed and mounted to a Folger Tech FT-5 R2 commercial 3D printer. The printhead uses a lead screw driven by a planetary-geared stepper motor to mechanically compress a disposable syringe, extruding cell suspension with sub-microliter resolution. The extruded cell suspension passes through the outer needle of a coaxial needle assembly during printing. A spool of collagen microfiber is loaded onto the printhead, which is fed through the inner needle of the coaxial needle assembly. At the outlet of the needle assembly, the collagen fiber is uniformly coated by the extruded cell suspension. The volume of cell suspension extruded per millimeter of drawn fiber is a user-determined process parameter and offers a means to control the resulting cell density throughout a scaffold. In preferred embodiments, cell density may vary from 0 to 10 million cells/mL depending on printing parameters and the desired number of cells within resulting scaffolds.

A custom micropost array consisting of two halves, each with two parallel rows of steel microposts (see FIG. 2), was designed as a receiving substrate. The microposts have a diameter of about 200 μm, inter-post spacing of about 1 mm on each half, and the distance between the two halves of the post array assembly is adjustable to facilitate fabrication of structures with different lengths.

The two halves are mounted to a bath, with the designed distance between them maintained. Prior to printing, the bath is filled with a crosslinking solution (for example, a thrombin solution), submerging the post array. During printing, the robotic printhead travels between the posts in a designed pattern. Collagen fiber is drawn out under tension by the posts and maintains the pattern traced by the printhead. An example pattern forming a single layer of fiber is shown in FIG. 3. The device is controlled to permit incremental adjustments to the vertical distance of the printhead from the micropost array, thereby causing repeating patterns as they are printed to form 3D structures.

The bath and post array assembly preferably are mounted onto manually adjustable linear and rotational stages. These stages are used for initial alignment of the printhead and post locations and to ensure that the orientation of the post array precisely aligns with the directional axes of the printer. After printing, constructs are removed from the micropost array by lifting them vertically.

To prevent fiber breakage at the tip of the coaxial needle, where a sharp angle is formed during printing, a thin-walled semi-flexible tubing preferably is fitted onto the exterior of the outer needle of the coaxial assembly with approximately 1 mm of tubing extended past the end of the needle. This allows for the fiber to be drawn out of the needle during printing without bending sharply at the rough edge of the steel needle. The semi-flexible tubing bends slightly, providing a softer surface and more gradual bend while still maintaining print precision.

In a preferred embodiment to print cellularized scaffolds, cells are suspended in a fibrinogen solution prepared in Dulbecco's Modified Eagles Medium (DMEM) and printed onto collagen fibers that are wound around microposts bathed in a thrombin solution that also is prepared in DMEM. Fibrinogen and thrombin, respectively, are an exemplary pair of a clinically relevant biocompatible hydrogel precursor and crosslinking solution. In the present example, the fibrinogen cell suspension rapidly solidifies to form a stable biocompatible fibrin gel when extruded into the thrombin crosslinking solution bath during printing. Fibrin gel has been widely used in FDA-approved medical procedures. A person skilled in the art will understand that the mechanical and bioactive properties of fibrin can be modulated by the user-determined concentrations of fibrinogen and thrombin.

The micropost array printing approach described herein can be practiced with a wide variety of cytocompatible reagent pairs that form a hydrogel when brought into contact with one another, such as a collagen solution printed into a neutralizing buffer or sodium alginate printed into an ionic crosslinking solution, to name a few. Other reagent pairs will be known to persons skilled in the art. In some embodiments, hyaluronic acid may be utilized as a cellular glue which facilitates the attachment of cells to the collagen fiber instead of fibrin, without the use of a crosslinking reagent. Other types of biological adhesives or biomaterials may also be used, such as Pluronic F127, gelatin, collagen, alginate, silk, etc. A person skilled in the art will understand that such biological glues and biomaterials possess appropriate adhesive and cohesive properties, such that they are able to facilitate the attachment of cells to the collagen fiber.

The Folger Tech FT-5 R2 hardware and firmware were modified to facilitate our printing approach. The commercial FDM printhead was removed and replaced with a custom extrusion printhead (FIG. 4). Non-stock components for the printhead and micropost array assembly were 3D printed in-house from PLA or machined. All stepper motors and drive pulleys were replaced to improve the resolution on the X, Y, and Z axes. The printer firmware was modified accordingly to accommodate these hardware changes.

A custom Python code was developed to accept user inputs for designed scaffold geometry and printing parameters and to output a corresponding g-code file. G-code is a common numerical control programming language, typically used to control automated computer aided manufacturing processes including 3D printing. User inputs to the Python code include the number of X-direction posts to wrap fiber around (determines the width of printed samples), diameter of posts, distance between the innermost rows of posts (determines the length of printed samples), number of fiber layers (determines the thickness of printed samples), vertical distance between fiber layers (the layer thickness), X-direction distance between posts, collagen fiber width, extruded filament diameter (determines volume of extruded cell suspension), printhead travel speeds for straight and circular segments, and cosmetic options for the graphical output of the resulting fiber pattern. The Python code calculates and outputs a g-code file, containing all parameters and motion/extrusion commands to execute a designed print, which is sent to the printer to produce the designed scaffold. Repetier-Host is used as a user interface to execute these commands as well as manual homing, motion, and extrusion commands. The Python code also outputs a true-to-scale graphical representation (example shown in FIG. 6) of the resulting fiber pattern and printhead motion path, which aids in construct design and troubleshooting.

The following is a description of exemplary scaffolds printed using the micropost array technique discussed herein and tested for strength and other properties.

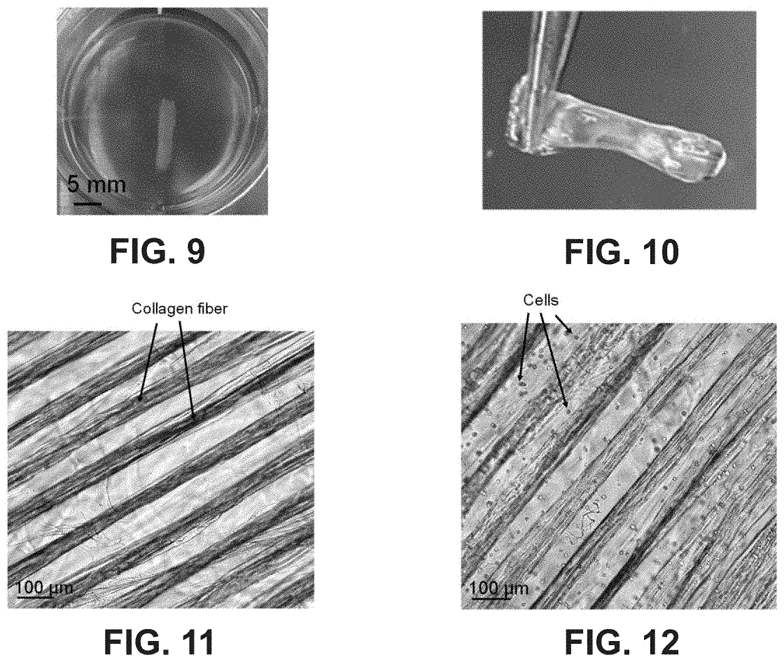

FIG. 9 is a macroscopic image of a printed scaffold submerged in media in a microwell plate. FIG. 10 is a macroscopic image of a printed scaffold held by forceps. FIG. 11 is a transmitted light microscopic image of an acellular printed scaffold including a pattern of parallel collagen microfiber segments. FIG. 12 is a transmitted light microscopic image of a cellular printed scaffold including a pattern of parallel collagen microfiber segments.

Composite hydrogel and collagen microfiber scaffolds were fabricated using the micropost array printing approach as described herein. Macroscopic views show that printed structures maintained their designed geometry when stored in media (FIG. 9) and when handled by tweezers (FIG. 10) after removal from the micropost array. Transmitted light microscopic views immediately after printing for structures printed without cells (FIG. 11) and with tenocytes harvested from rat tail (FIG. 12) revealed evenly spaced, highly parallel fiber segments. For cellularized scaffolds, cells are suspended in fibrin gel with a uniform distribution between parallel collagen microfiber segments.

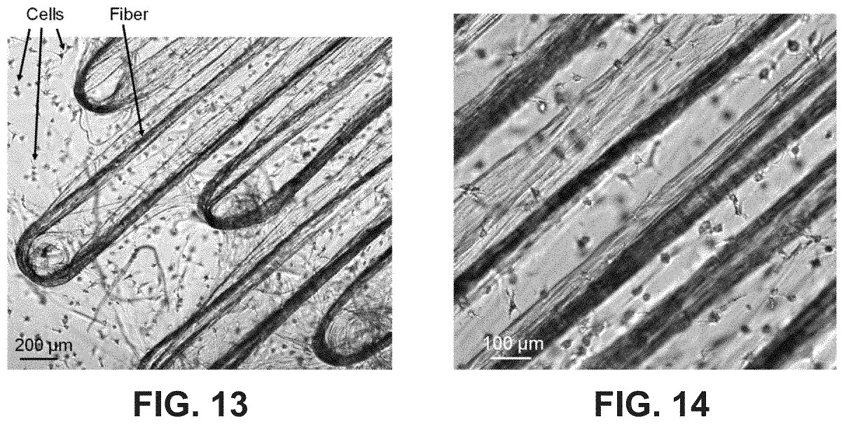

FIG. 13 is a microscopic image of a scaffold pattern formed by wrapping a collagen fiber around a micropost array after two days of standard cell culture conditions. FIG. 14 is the microscopic image from FIG. 13 at twice the magnification of FIG. 13, showing visibly extended cell morphology indicating interaction with the hydrogel environment.

The unique pattern formed by wrapping fiber around alternating microposts within the array was maintained after 2 days under typical cell culture conditions (FIG. 13). Cell morphology became visibly more extended over 2 days of culture (FIGS. 13 and 14) as compared to the substantially spherical morphology of the cells that were visible immediately after printing (FIG. 12), indicating cell-material interaction (extension, attachment, migration) with the bioactive fibrin environment. Printed scaffolds were found to maintain the original fiber patterns for several weeks in culture. Notably, it was observed for cellularized scaffolds that the fibrin gel contracted to more tightly surround the collagen fiber segments during extended culture periods. It is believed that this is due to cellular forces present when culturing tenocytes within the soft compressible matrix.

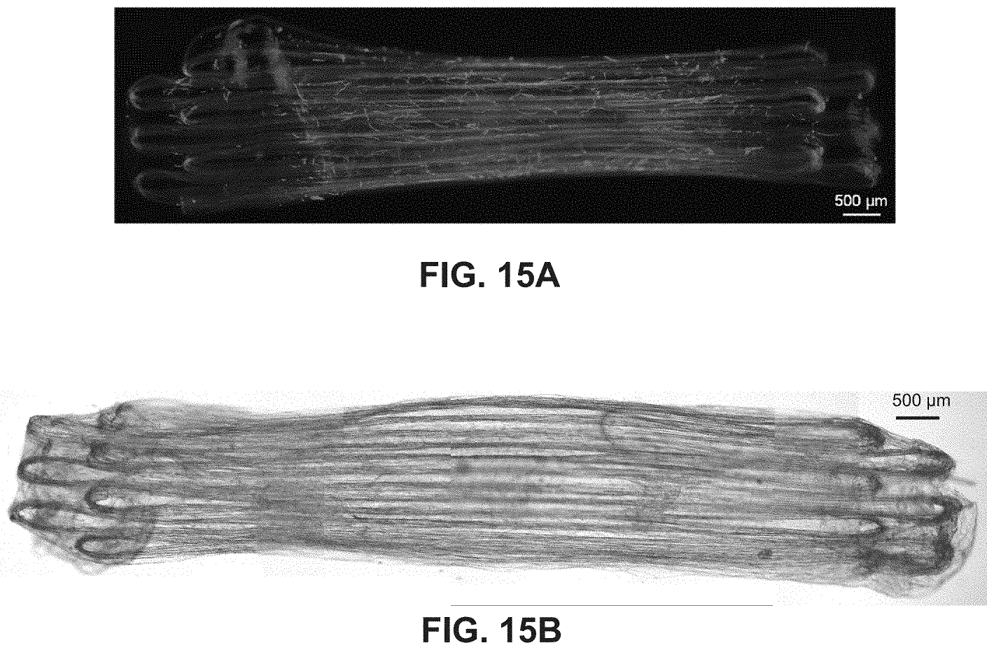

FIG. 15A is a fluorescence image of a scaffold after seven days of culture, showing cytoskeleton protein vimentin, cell nuclei using DAPI, and collagen fiber autofluorescence at 495 nm. FIG. 15B is a composite image from 12 fields of view of a printed cellular construct after 7 days of culture.

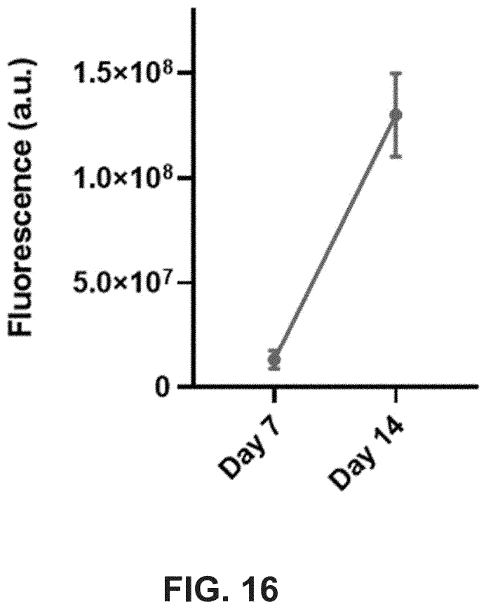

FIG. 16 is a graph showing the results of an alamarBlue assay indicating metabolic activity for scaffolds printed with human tenocytes after 7 and 14 days of culture.

Fluorescence imaging of scaffolds printed with human tendon cells, or tenocytes (ZenBio), after 7 days of bioreactor culture under constant tension reveals a relatively uniform distribution of cells throughout, visualized by labeling the cytoskeletal protein vimentin (FIG. 15). Parallel collagen microfiber maintaining the distinct pattern formed from the micropost array is visible due to the fiber's autofluorescence at 495 nm. Notably, cells are largely absent from the left and right ends of the scaffold where the fibrin gel was displaced by compression clamps which compressed the ends of the scaffold for culture in the bioreactor. Additionally, scaffolds were printed with human tenocytes to assess cell metabolic activity over time using the alamarBlue assay while cultured under constant tension. Samples were removed from bioreactor chambers after 7 days and 14 days of culture. At each timepoint, scaffolds were incubated for 4 hours in 10% alamarBlue solution in tenocyte growth media and fluorescence was measured according to standard protocols. Metabolic activity of cellularized scaffolds in culture (n=4) was found to increase from 7 to 14 days of culture (FIG. 16), indicating an increase in cell health, activity, and proliferation.

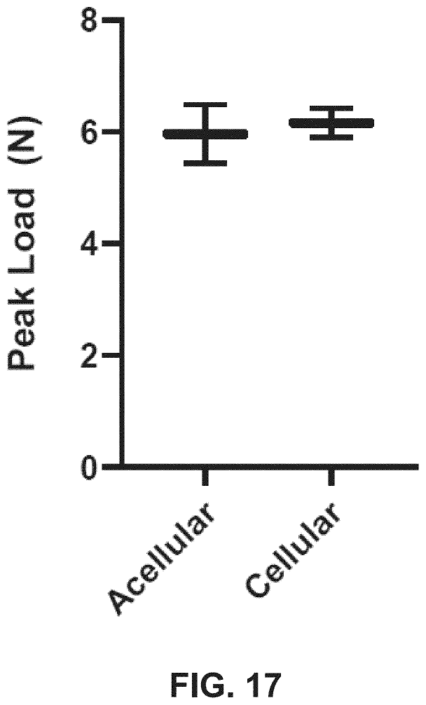

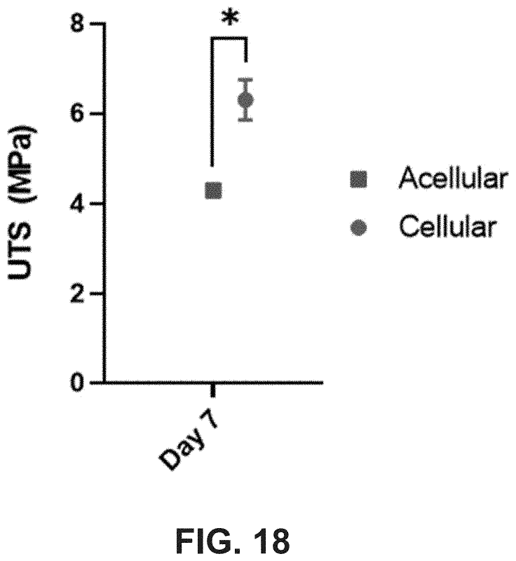

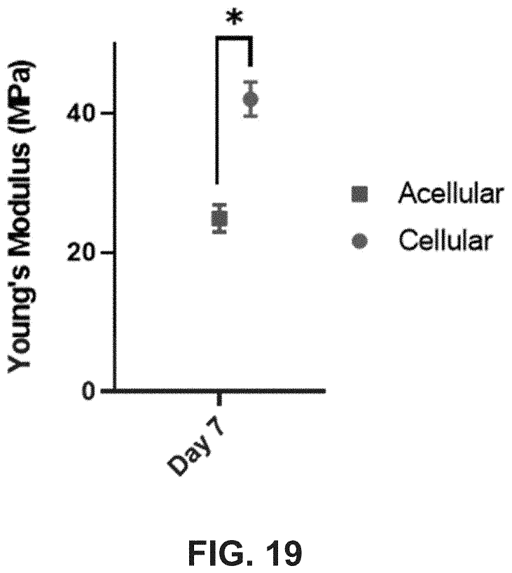

Composite scaffolds were printed with and without cells using the micropost array approach and their mechanical properties were assessed. FIG. 17 is a graph showing peak load of acellular and cellular composite collagen microfiber scaffolds printed using the micropost array approach after seven days of constant-tension bioreactor culture. FIG. 18 is a graph showing ultimate tensile strength (UTS) of acellular and cellular composite collagen microfiber scaffolds printed using the micropost array approach after seven days of constant-tension bioreactor culture. FIG. 19 is a graph showing Young's modulus of acellular and cellular composite collagen microfiber scaffolds printed using the micropost array approach after seven days of constant-tension bioreactor culture.

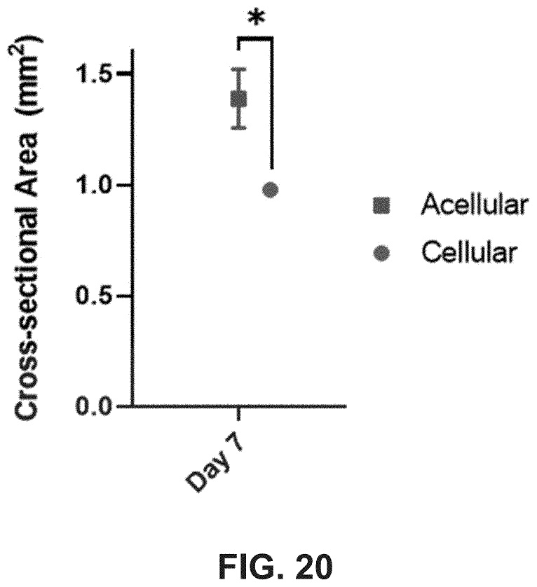

FIG. 20 is a graph showing cross-sectional area of acellular and cellular composite collagen microfiber scaffolds printed using the micropost array approach after seven days of constant-tension bioreactor culture.

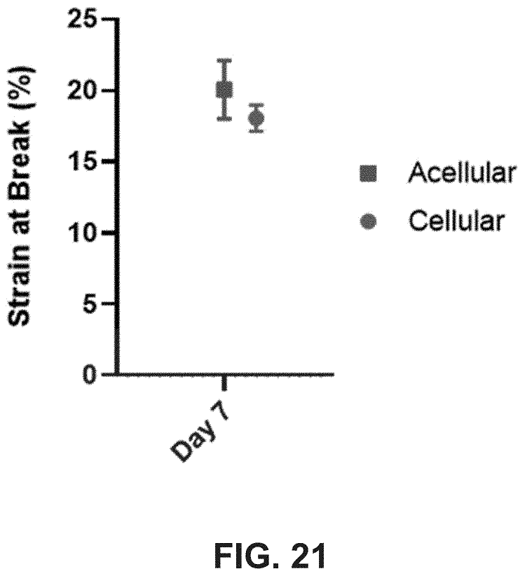

FIG. 21 is a graph showing strain at break of acellular and cellular composite collagen microfiber scaffolds printed using the micropost array approach after seven days of constant-tension bioreactor culture.

As a pilot study, two acellular scaffolds were fabricated and two scaffolds were fabricated with rat tail tenocytes. For both acellular and cellular scaffolds, fibrinogen was prepared in DMEM to 40 mg/mL and the thrombin bath solution was prepared in DMEM to 0.2265 mg/mL. For cellular scaffolds, cells were suspended in the fibrinogen solution to 750,000 cells/mL.

Both scaffolds were cultured under constant tension within a bioreactor for 7 days. Immediately before testing, samples were removed from culture and excess media was removed using a lint-free wipe. Sample cross sections were measured using calipers. Each end was clamped into grips of a uniaxial tensile testing machine (MTS Systems Corporation, Eden Prairie, Minn.) with a 100 N load cell. Samples were pulled to failure with a grip displacement speed of 0.5 mm/sec and load and displacement data were recorded. Young's modulus (E) was determined by the linear region of the stress-strain curve and ultimate tensile strength (UTS) was determined using the highest recorded load. Peak load, UTS, and Young's modulus are shown in FIGS. 17, 18, and 19, respectively, for acellular and cellular scaffolds tested to failure. From this pilot study, no significant difference was found between the mechanical properties of acellular and cellular scaffolds.

### Treatment of Volumetric Muscle Loss

Musculoskeletal tissue injuries, including volumetric muscle loss (VML), are commonplace and often lead to permanent disability and deformation. One aspect of the present invention relates to the preparation and use of cellularized collagen microfiber implants to facilitate functional repair and regeneration of such musculoskeletal soft tissues. For similar collagen microfiber scaffolds to those described above, clinically relevant cells were positioned controllably along clinically relevant, high strength collagen fibers to biomanufacture musculoskeletal tissue analogs for restoring form and function to injured tissues. Accordingly, the scaffold manufacturing methods described herein may be utilized to form scaffold constructs for use in treating volumetric muscle loss (VML). Below is a description of the VML treatment procedure and details regarding testing of such a procedure using scaffolds similar to those described above.

Human mesenchymal stem cells (hMSCs) or rat muscle progenitor cells (MPCs) are bioprinted to create an engineered implant that may be valuable for a diverse array of indications including, tendon or muscle regeneration. Mesenchymal stem cells offer excellent potential for augmenting musculoskeletal tissue repair and regeneration due to their immune-evasive properties [Ankrum 2014, Zhang 2015], therapeutic effects [Zhang 2015, Jang 2015, Lee 2017], multilineage differentiation potential [Pittenger 1999], and availability as a commercial clinically relevant cell type. Similarly, MPCs have shown marked therapeutic effects in facilitating functional recovery in volumetric muscle loss injuries in validated animal models [Mintz (2020), Passipieri (2019)].

The present invention is based in part on the discovery that glyoxal crosslinked collagen fibers with high tensile strength can be used as a filament for bioprinting and can recreate the structural, cellular, and mechanical likeness of native tissue in an automated, scalable fabrication process, which was previously an ambitious and unrealized challenge [Murphy (2014, Murphy 2020)].

Results using implants produced using an Assembled Cell—Decorated Collagen (“AC-DC”) bioprinting process showed that the directionality and distribution of cells throughout implants mimic the cellular properties of native musculoskeletal tissue. Bioprinted implants according to the invention approximate and can be adjusted to exceed the strength and stiffness of human musculoskeletal tissue. Moreover, they exceeded the properties of commonplace collagen hydrogels by orders of magnitude.

The regenerative potential of such implants was also assessed in vivo in a rodent VML model. A critically sized muscle injury in the hindlimb was created and repaired, and limb torque generation potential was measured over 12 weeks. Both acellular and cellular implants were found to promote functional recovery compared to the unrepaired group, with AC-DC implants containing therapeutic muscle progenitor cells promoting the highest degree of recovery.

Histological analysis and automated image processing of explanted muscle cross-sections revealed increased total muscle fiber count, median muscle fiber size, and increased cellularization for injuries repaired with cellularized implants. These studies introduce the tremendous potential of an advanced bioprinting method for generating tissue analogs with near native biological and biomechanical properties with the potential to repair numerous challenging musculoskeletal injuries.

### Example

### Functional Recovery in a VML Model

In vivo skeletal muscle repair studies were conducted over 12 weeks in a validated rodent VML model using implants similar to those described above. Details of these studies are available at K. W. Christensen, J. Turner, K. Coughenour, Y. Maghdouri-White, A. A. Bulysheva, O. Sergeant, M. Rariden, A. Randazzo, A. J. Sheean, G. J. Christ, M. P. Francis, “Assembled Cell-Decorated Collagen (AC-DC) bioprinted implants mimic musculoskeletal tissue properties and promote functional recovery,” published Jul. 2, 2021, and available pre-print via bioRxiv at: https://doi.org/10.1101/2021.06.22.449431. The entire disclosure of this publication is incorporated herein by reference.

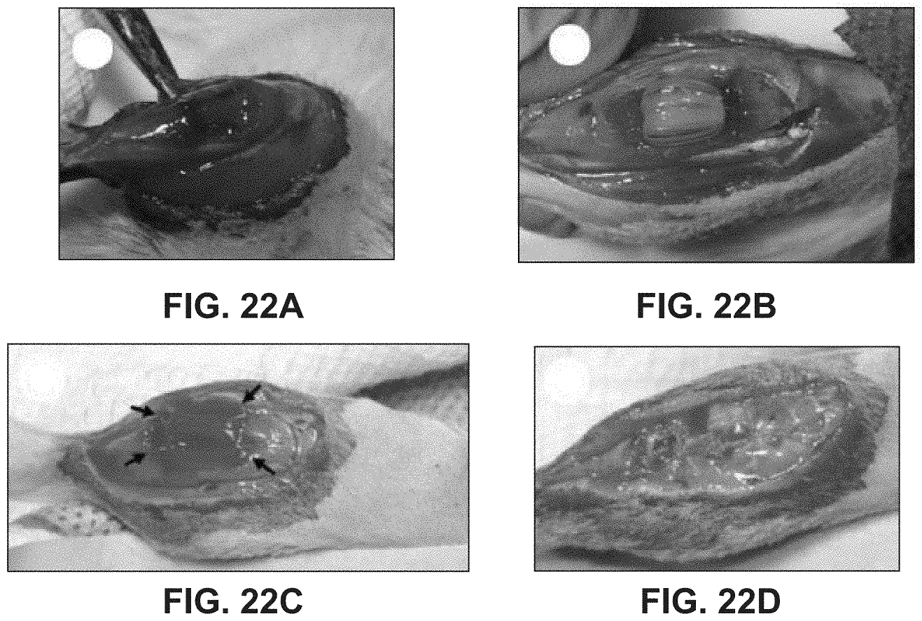

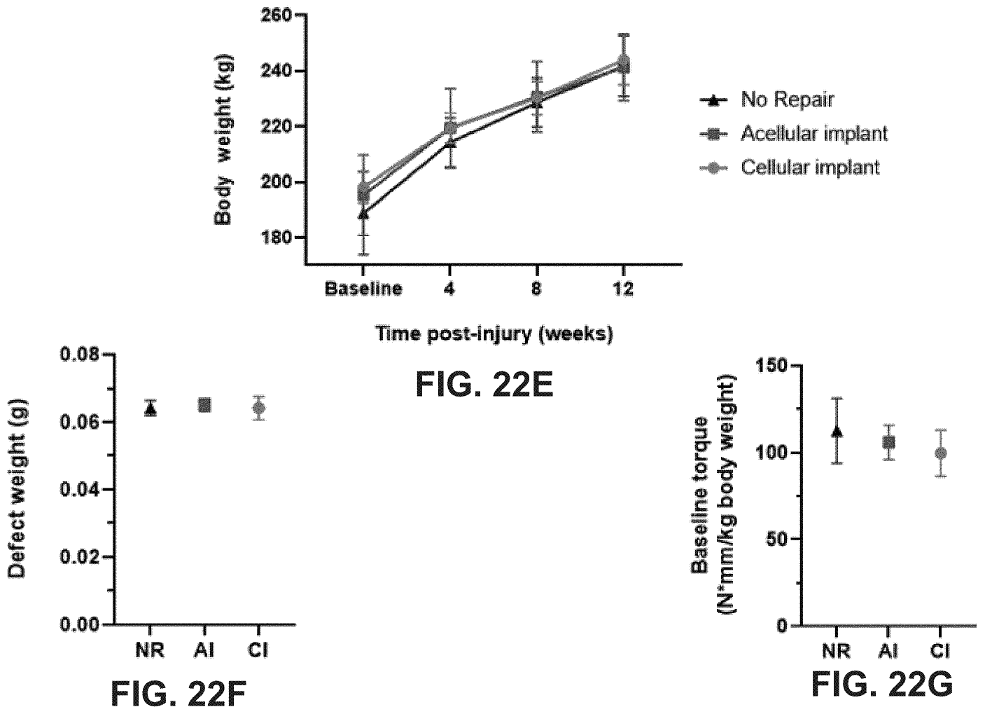

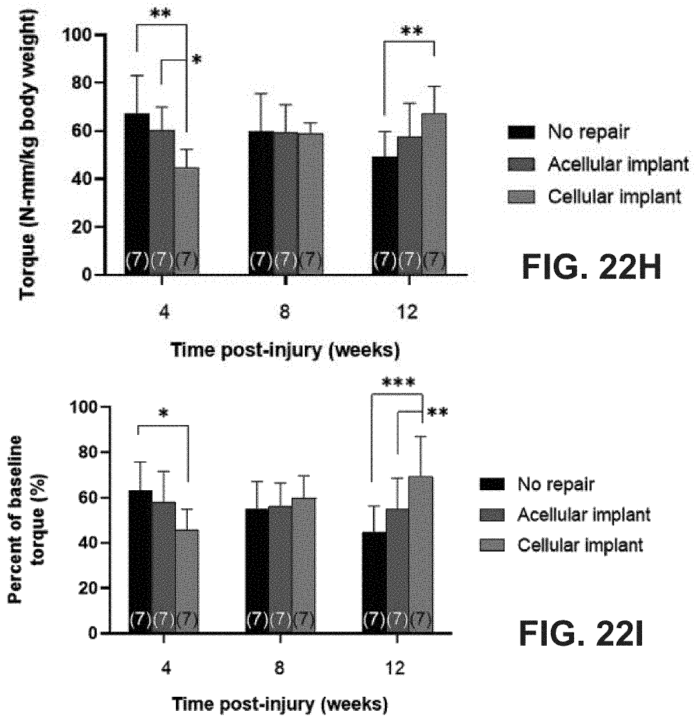

At least 20% of overall muscle weight was removed from the tibialis anterior (TA) muscle of the lower left hindlimbs of Lewis rats [Mintz (2020), Corona (2014)]. Three methods of repair were assessed head-to-head: a control group receiving no repair, an acellular implant group receiving repair with AC-DC implants with no cellular component, and a cellular implant group receiving repair with AC-DC implants printed with rodent MPCs. Defect creation, initial placement of an implant, suture placement for implant attachment, and fascia replacement are shown in FIG. 22A-D, respectively. Specifically, FIG. 22A shows creation of a VML injury measuring approximately 1 cm×0.7 cm×0.5 cm and weighing at a minimum 20% of the overall TA weight. FIG. 22B shows an acellular AC-DC implant inserted into the injury site, which, in FIG. 22C is sutured into the injury site with arrows indicating attachment points. FIG. 22D shows fascia sutured overtop of the injury site to secure the implant in place further.

All animals recovered post-surgery, and there were no signs of infection and no deaths. Across experimental groups, animal body weight increased similarly over the 12-week period (FIG. 22E), which shows animal weight pre-injury and at 4, 8, and 12-weeks postinjury, corresponding to functional testing timepoints. Measured defect weight at the time of surgery was not statistically different, as shown in FIG. 22F, which presents the weight of defects created for “no repair,” “acellular implant,” and “cellular implant” (NR, AI, and CI, respectively) experimental groups (p=0.8, no significant difference). In FIGS. 22E to I, all data is based on n=7 per group per time point (*p<0.05 indicates significance).

Functional testing was performed in vivo before defect creation and at 4, 8, and 12-weeks post-repair to assess muscle recovery post-operatively. Briefly, rat hind limbs were attached to a motorized footplate and stimulated electrically to measure maximum isometric torque generation [Mintz (2020), Passipieri (2019), Corona (2014)]. Mean values are expressed as torque normalized to animal body weight at each time point (N-mm/kg of body weight) to control for increases in torque production due to animal growth. Baseline torque generation capability before defect creation did not vary statistically between treatment groups, as shown in FIG. 22G (p=0.9, no significant difference. Torque generation post-repair is expressed as raw torque (FIG. 22H) and percent of baseline torque generation is shown in FIG. 22I. Measured torque and percent of baseline torque is shown at 4, 8, and 12 weeks post-repair. These figures indicate that functional recovery is facilitated by implant implantation. Both methods show similar trends with only slight variations in statistical significance.

Most notably, significant improvements in torque generating capability were observed over 12 weeks for injuries repaired with cellularized implants containing therapeutic MPCs. At 4 weeks, raw torque generation was significantly lower in the acellular and cellular implant groups than no repair, and the percent of baseline torque was significantly lower in the cellular implant group. This initial decrease in torque generation capabilities is believed to be due to the early wound healing processes, or possibly related to the initial tensile properties of the implant. However, by 8 weeks post-repair, there was no difference observed between the treatment groups.

At 12-weeks post-repair, in contrast to findings at 4 weeks, raw torque generation was found to be significantly higher in the cellular implant group compared to the no repair group, and the percent of baseline torque was significantly higher in both the acellular and cellular implant groups, revealing key trends in the functional recovery of a VML injury among treatment groups. In addition, significant deterioration of function was found over 12 weeks for animals receiving no repair. In contrast, torque generation remained largely consistent for animals repaired with acellular implants, indicating that the presence of the collagen fiber implant without cells attenuated the functional deterioration associated with no repair.

Notably, the ablation of synergistic muscles during defect creation removes ˜20% of torque generation in the anterior compartment [Mintz (2020)]. As such, normalized torque would be limited to ˜85 N-mm/kg across the treatment groups (106 N-mm/kg average at baseline). The mean functional recovery of the cellularized implant group at 12 weeks was 76% of the maximum theoretical recovery following synergist ablation compared to 67% in the acellular group and 57% in the no repair group. In addition, three of the seven animals receiving repair with cellular implants were observed to have a functional recovery of greater than 87%, with one animal recovering to near-maximal theoretical recovery compared to preinjury levels (99%).

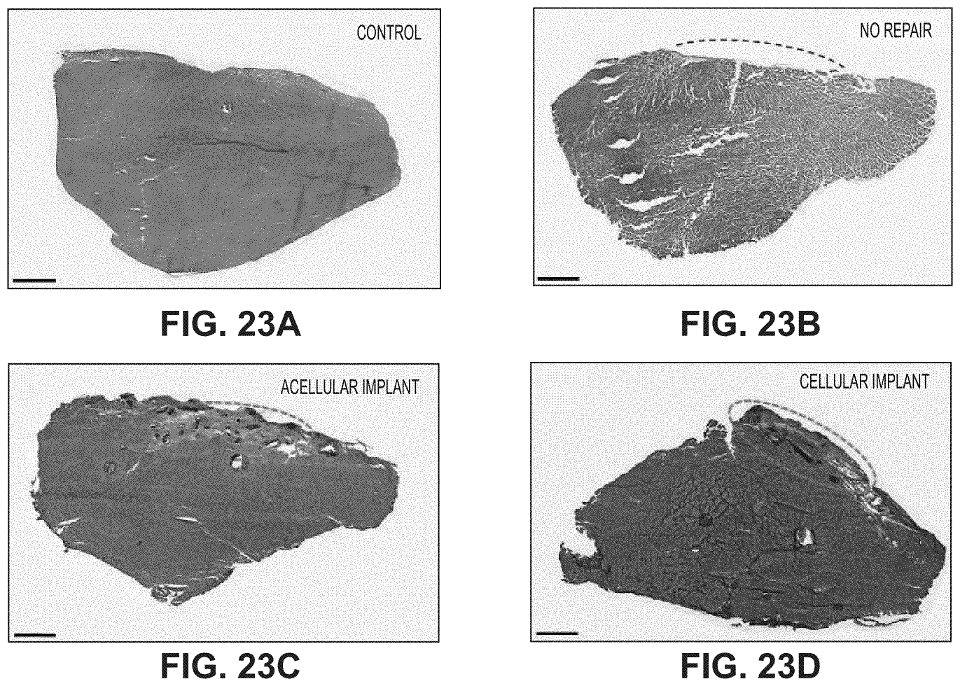

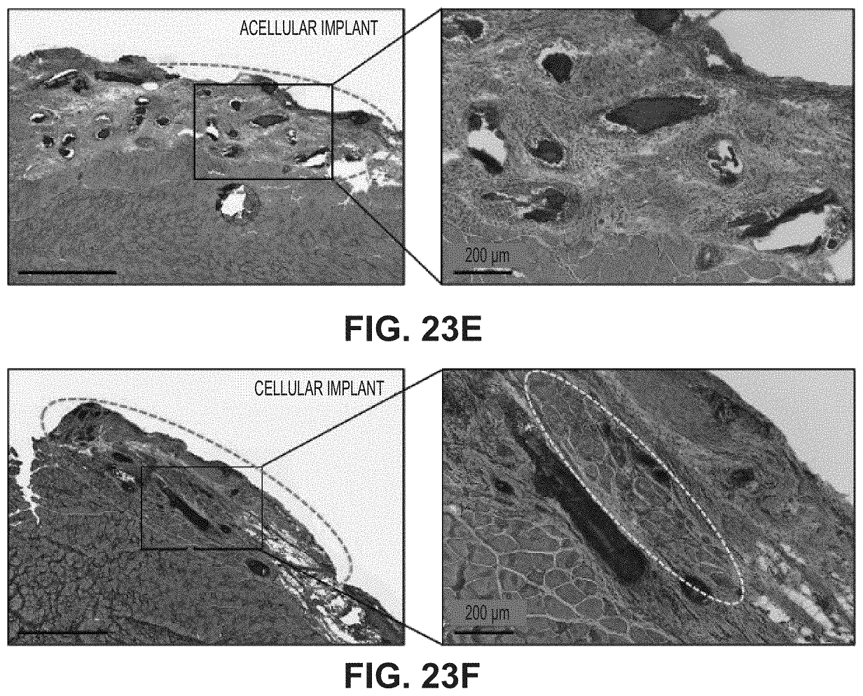

Following assessment of functional recovery in vivo at 12-weeks, isolated TA muscles were collected for morphological and histological examination. The gross morphology of those repaired by acellular and cellular AC-DC implants appeared more similar to control muscles than did the no repair group, which exhibited convex indentations at the injury location. More fascia was also noted in the repair groups. The distinction between implants and surrounding tissue was not obvious, indicating tissue ingrowth around or resorption of the collagen fiber implants. Isolated muscles were cross-sectioned through the belly and processed for H&E staining, with representative images for each experimental group shown in FIGS. 23A-D.

In FIG. 23A-D, representative H&E images of the tibialis anterior (TA) muscle are shown for (A) uninjured control, (B) no repair, (C) acellular implant, and (D) cellular implant experimental groups after 12 weeks. A black dashed line indicates the approximate area of defect creation. Green dashed ovals identify AC-DC implant locations.

In FIG. 23E-F, magnified views of (E) acellular implant and (F) cellular implant locations with magnified windowed views showing cellular ingrowth and muscle fiber formation in the cellular implant location (yellow dashed oval). All scale bars in FIG. 23 are 1 mm unless otherwise noted.

As with gross examination, the unrepaired group exhibited distinct depressions at the injury site indicating a lack of tissue regeneration (FIG. 23B). Animals repaired with acellular and cellular implants, in contrast, exhibited more fullness to the tissue and uniform cross-sections similar to uninjured controls and thus improved cosmesis. Collagen fiber remaining from implants is visible within the injury sites as deep pink somewhat-circular cross-sections on the order of 100 μm diameter. Cellular ingrowth is visible in and around the implants (FIGS. 23E and 23F). Fiber cross-sections are more apparent in the acellular implant group than the cellular implant group, possibly indicating an increased rate of fiber resorption for cellularized implants. For injuries repaired with cellular AC-DC implants, the presence of new muscle fibers at the implant site was noted (FIG. 23F).

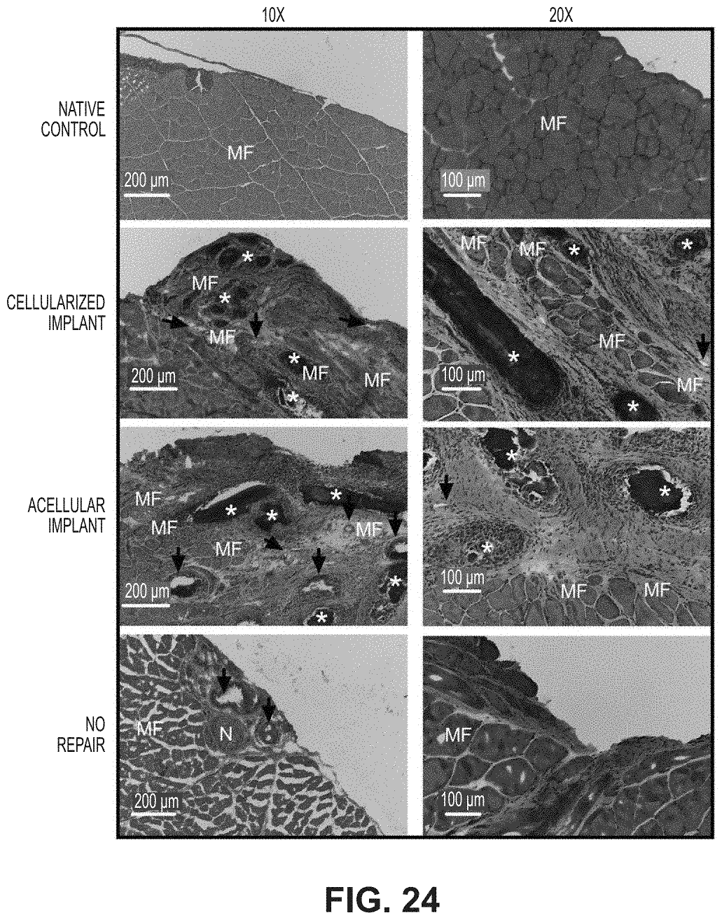

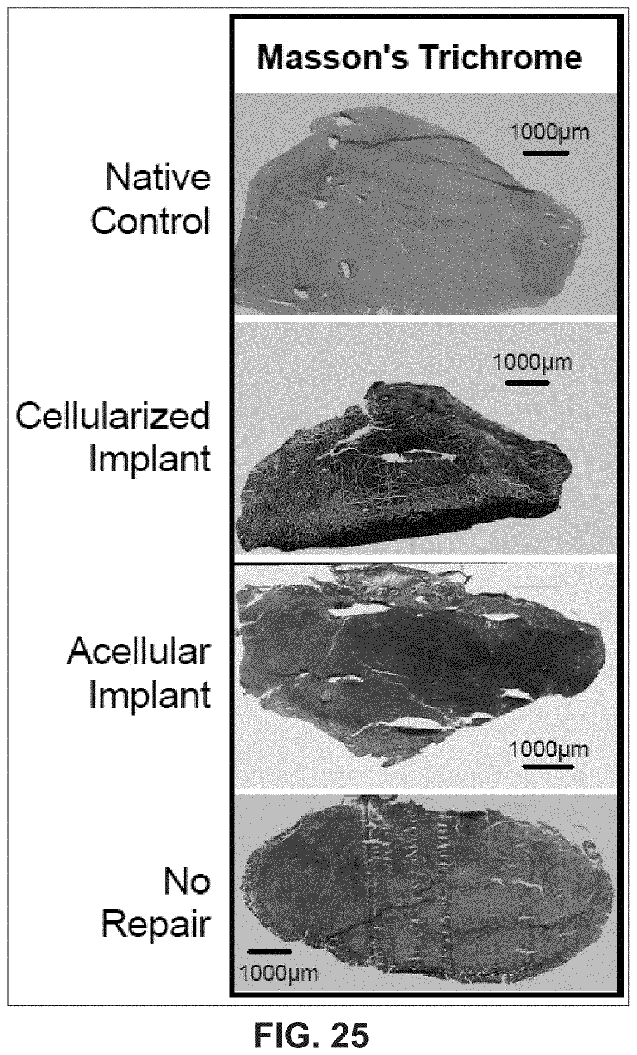

Higher magnification images from the H&E section further revealed the new muscle fibers and angiogenesis within the implant region FIG. 24 along with new collagen deposition as qualitatively indicated by Masson's Trichrome staining around the implant region in FIG. 25. In FIG. 24, prominent new muscle fibers are labeled with “MF” and were found growing within the AC-DC implants as demarcated by the implant collagen fibers (labeled with “*”). Nearby blood vessels are also noted prominently in the implant region and are called out with a black arrow. Nerve bundles are noted with “N.” In FIG. 25, a section from each group show the presence of collagen within and around the implant region and defect area.

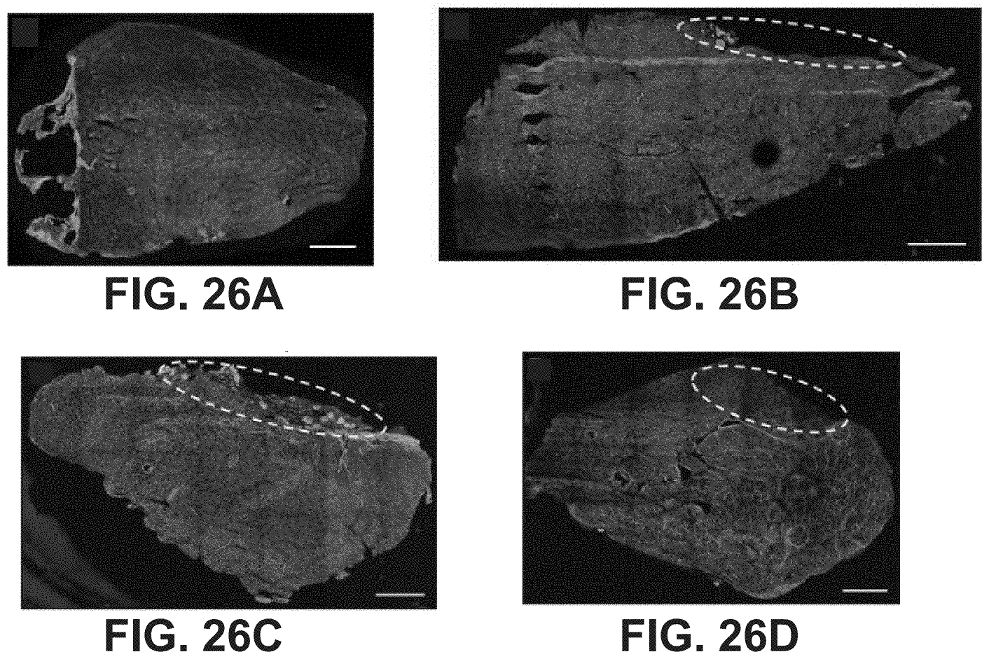

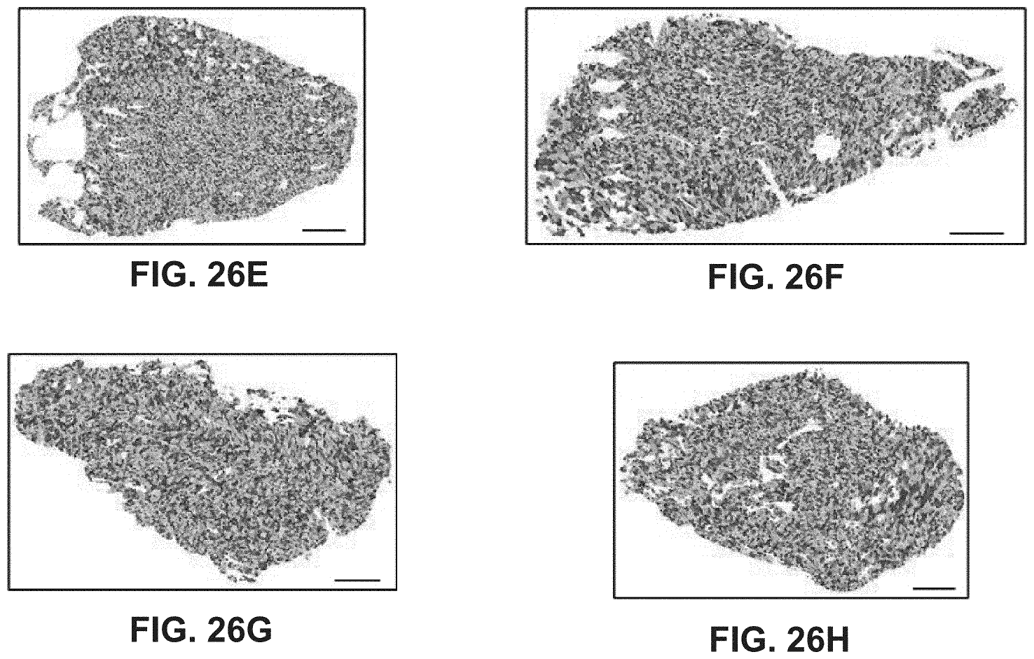

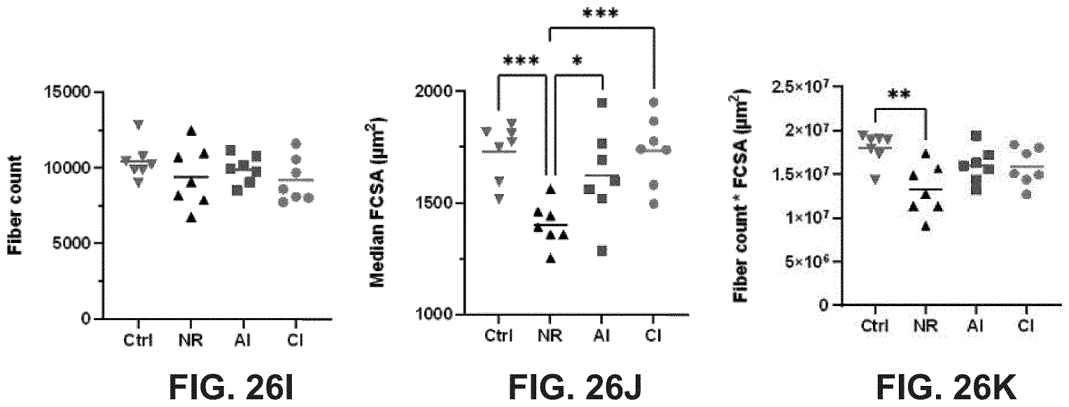

Additional sections from the TA muscle belly were processed for analysis using SMASH, a semi-automated muscle fiber analysis software as shown in FIGS. 26A-K. In FIGS. 26A-D, representative laminin-stained sections of the TA muscle are shown for (A) uninjured control, (B) no repair, (C) acellular implant, and (D) cellular implant experimental groups with dashed ovals indicating the approximate region of injury. In FIGS. 26E-H, colorized outputs from the software identifying individual muscle fibers within sections corresponding to (FIGS. 26A-D), respectively. FIG. 26I shows total fiber count, FIG. 26J shows median fiber cross-sectional area (FCSA), and FIG. 26K shows the product of fiber count and FCSA for uninured control (Ctrl), no repair (NR), acellular implant (AI), and cellular implant (CI) experimental groups. All scale bars in FIG. 26 are 1 mm. The data presented are based on n=7 per group per time point, and *p<0.05 indicates significance.

Referring to FIG. 26, laminin and fluorophore 488 staining identify the outline of muscle fibers throughout sections (FIG. 26A-D) and SMASH analysis allows for individual fiber distinction, as seen with colorization applied (FIG. 26E-H). Analysis of the total number of fibers yields no significant difference between the uninjured control, no repair, acellular implant, and cellular implant groups (FIG. 26I). However, the median fiber cross-sectional area (FCSA) in muscle sections repaired with acellular and cellular AC-DC implants was significantly larger than that of the no repair group and did not differ significantly from the uninjured control (FIG. 26J). The cellularized implant and control groups show the greatest difference from the no repair group, with p values of 0.0007 and 0.0002, respectively.

Multiplying the total number of fibers by the median fiber cross-sectional area offers a representation of the total muscle fiber cross-sectional area (FIG. 26K). Again, this product shows no significant difference between uninjured controls and injuries repaired with acellular and cellular implants after 12 weeks in life, supporting that AC-DC implants facilitated an increase in total muscle fiber area.

To summarize, a method of treating volumetric muscle loss (VML) may include affixing, within a VML wound site, a scaffold construct formed of a plurality of collagen microfibers arranged in an organized sheet-like configuration and coated with a biocompatible solution. In some cases, affixing the scaffold construct within the VML wound site may include suturing two or more portions of the scaffold construct to muscle tissue within the VML wound site. For example, four corners of the scaffold construct may be sutured within the wound site. In addition, in some cases, the method may further include replacing the fascia over the scaffold construct after affixing the scaffold construct within the VML wound site.

It will also be noted that, in some cases, the scaffold construct implanted within a given VML wound site may include multiple sheet-like layers of the collagen microfibers. The number of layers utilized may vary depending on the depth of the wound site. Similarly, the overall size of the scaffold construct used may vary depending on the size of the wound site.

In some cases, the biocompatible solution with which the collagen microfibers is coated may be hyaluronic acid. In some cases, the biocompatible solution may be a cell suspension. In such cases, the cell suspension may include muscle progenitor cells (MPC's). The cell suspension may include approximately 4,000,000 cells/mL. Other concentrations could alternatively be used to form the scaffold construct.

The scaffold construct implanted to treat VML may have mechanical properties substantially approximating or exceeding those of human tendon. Notably, the scaffold construct may have a mean ultimate tensile strength (UTS), tensile modulus, and strain at break that substantially approximate or exceed those of human tendon.

### Preferred Scaffold Constructs:

Preferred embodiments of the scaffold constructs according to the invention will have a length dimension and a width dimension independently ranging from about 1 mm to 10 mm, depending on the size and shape of the damaged area for which a repair is intended. Preferably, the length and width of a scaffold construct to be used for VML will independently be about 2 cm to 9 cm, 3 cm to 8 cm or 4 cm to 7 cm. Alternative embodiments may be standardized in a 4 cm (width) by 10 cm (length) construct, more preferably 6 cm by 10 cm, 8 cm by 10 cm and 10 cm by 10 cm.

The thickness of a scaffold construct according to the invention may be limited by the ability of the recipient host's surrounding tissue to vascularize the implant such that the cells adhered to the construct remain viable. Accordingly, preferred scaffold constructs will be about 0.5 mm, 1 mm, 1.5 mm, 2 mm, 2.5 mm, 3 mm, 3.5 mm, 4 mm, 4.5 mm, and 5 mm in depth (thickness). In preferred constructs, the implant is produced by forming about 2 to 8 layers of dispensed and coated fiber.

For purposes of implantation, multiple scaffold constructs may be used by a surgeon in the repair of VML. These constructs may be stacked or arranged sequentially along an area for which repair is intended.

The printhead may be configured to produce scaffold constructs in which the spacing between fibers may be adjusted as discussed above. For example, the spacing between fibers preferably will range on average from about 0 (that is, the fibers are directly adjacent) to about 1 mm. In preferred embodiments, the average spacing between fibers will be about 100, 200, 300, 400, 500, 600, 700, 800, 900 and 1,000 microns.

The preparations of cell suspensions according to the invention will be sufficiently dense to effectively coat the dispensed fibers of a scaffold construct. Preferred cell densities range from about 0 to about 10 million cells/mL, as discussed above. Preferred cell suspension densities will be about 100,000, 200,000, 400,000, 600,000, 800,000, 1 million, 1.5 million, 2 million, 3 million, 4 million, 5 million, 6 million, 7 million, 8 million, 9 million and 10 million cells/mL. Preferably, about 0.1 to 10 microliters of cells suspension are extruded per millimeter of drawn fiber for preferred embodiments.

For the cells populations that are adhered to the scaffold constructs, preferred numbers of cells on the construct will range from about 100,000 to about 1 million cells per implant or more than 1 million cells per implant. Preferred ranges are about 200,000 to 900,000, 300,000 to 800,000, 400,000 to 700,000 and 500,000 to 600,000 cells per implant.

Preferred hydrogels, as described above, should stabilize rapidly, with solidification beginning within seconds of contact with a crosslinking solution.

While various embodiments of the present invention have been described, the description is intended to be exemplary, rather than limiting, and it will be apparent to those of ordinary skill in the art that many more embodiments and implementations are possible that are within the scope of the invention. Although many possible combinations of features are shown in the accompanying figures and discussed in this detailed description, many other combinations of the disclosed features and methods are within the ordinary level of skill in this field. Any feature of any embodiment may be used in combination with or substituted for any other feature or element in any other embodiment unless specifically restricted. Therefore, it will be understood that any of the features shown and/or discussed in the present disclosure may be implemented together in any suitable combination. Accordingly, the embodiments are not to be restricted except in light of the attached claims and their equivalents. Also, various modifications and changes may be made as desired by a person skilled in the art and these remain within the scope of the attached claims.

### REFERENCES

Abrahamsson (2010) “Chondrogenesis and mineralization during in vitro culture of human mesenchymal stem cells on three-dimensional woven scaffolds”

Ghorbanian (2014) “Microfluidic direct writer for cell-laden hydrogel constructs”

Kaiser (2019) “Digital design and automated fabrication of bespoke collagen microfiber scaffolds”

Liberski (2011) “Organ weaving —woven threads and sheets as a step towards artificial organ development”

Mandrycky (2016) “3D bioprinting for engineering complex tissues”

Mauck (2009) “Mechanics of nanofibrous assemblies for fiber-reinforced tissue regeneration”

Merceron (2015) “3D bioprinted complex structure for engineering muscle-tendon unit”

O'Keefe (2019) “American society for bone and mineral research report on cell-based therapies”

Onoe (2011) “Living cell fabric”

Sahoo (2007) “Hybrid polymer scaffolds for potential ligament and tendon tissue engineering”

Sugimoto (2011) “Implantable hydrogel microfiber encapsulating pancreatic cells for diabetes treatment”

Tamayol (2013) “Fiber-based tissue engineering progress, challenges, and opportunities”

Walters (2012) “Design and analysis of braid-twist collagen scaffolds”

Wu (2015) “Direct E-jet printing of 3D fibrous scaffold for tendon tissue engineering”

Skardal (2014) “Biomaterials for Integration with 3-D Bioprinting”

Mintz (2020) “Long-Term Evaluation of Functional Outcomes Following Rat Volumetric Muscle Loss Injury and Repair”

Murphy (2014) “3D bioprinting of tissues and organs”

Murphy (2020) “Opportunities and challenges of translational 3D bioprinting”

Itoi (1995) “Tensile properties of the supraspinatus tendon”

Ankrum (2014) “Mesenchymal stem cells: immune evasive, not immune privileged”

Zhang (2015) “The challenges and promises of allogeneic mesenchymal stem cells for use as a cell-based therapy”

Jang (2015) “Efficacy and Safety of Human Umbilical Cord Blood—Derived Mesenchymal Stem Cells in Anterior Cruciate Ligament Reconstruction of a Rabbit Model: New Strategy to Enhance Tendon Graft Healing”

Lee (2017) “Therapeutic Mechanisms of Human Adipose-Derived Mesenchymal Stem Cells in a Rat Tendon Injury Model”

Pittenger (1999) “Multilineage Potential of Adult Human Mesenchymal Stem Cells”

Passipieri (2019)“In Silico and In Vivo Studies Detect Functional Repair Mechanisms in a Volumetric Muscle Loss Injury”

Foolen (2018) “Tissue alignment enhances remodeling potential of tendon-derived cells—Lessons from a novel microtissue model of tendon scarring”

Wang (2003) “Cell orientation determines the alignment of cell-produced collagenous matrix”

Han (2014) “Lagrangian analysis of consecutive images: Quantification of mixing processes in drops moving in a microchannel”

Ober (2015) “Active mixing of complex fluids at the microscale”

Chandrashekar (2006) “Sex-based differences in the tensile properties of the human anterior cruciate ligament”

Achilli (2010) “Tailoring Mechanical Properties of Collagen-Based Scaffolds for Vascular Tissue Engineering: The Effects of pH, Temperature and Ionic Strength on Gelation”

Taylor (2013) “In vivo measurement of ACL length and relative strain during walking”

Corona (2014) “Implantation of In Vitro Tissue Engineered Muscle Repair Constructs and Bladder Acellular Matrices Partially Restore In Vivo Skeletal Muscle Function in a Rat Model of Volumetric Muscle Loss Injury”

What is claimed is:

1. A scaffold construct, comprising:
a biocompatible hydrogel; and
at least one biomaterial microfiber strand wound to form a plurality of microfiber segments in proximity to one another and arranged in an organized configuration.

2. The scaffold construct of claim 1, wherein, in the organized configuration, at least some of the plurality of microfiber segments are aligned substantially parallel to one another.

3. The scaffold construct of claim 1, wherein, in the organized configuration, at least some of the plurality of microfiber segments are arranged oblique to one another.

4. The scaffold construct of claim 1, wherein the plurality of microfiber segments includes multiple layers of microfibers stacked to form a three-dimensional construct.

5. The scaffold construct of claim 1, wherein the biomaterial is selected from the group consisting of collagen, elastin, hyaluronic acid, fibrinogen, fibrin, fibronectin, silk, alginate, and pluronic.

6. The scaffold construct of claim 5, wherein the biomaterial is collagen.

7. The scaffold construct of claim 6, wherein the biocompatible hydrogel further comprises cells distributed therein.

8. The scaffold construct of claim 1, wherein the biocompatible hydrogel is crosslinked.

9. An apparatus for making a scaffold construct, the apparatus comprising:
a first array of microposts; and
a second array of microposts arranged spaced from the first array of microposts;
wherein the microposts are configured to receive a microfiber strand to form a plurality of segments, wherein at least some of the plurality of segments are arranged in a substantially aligned configuration.

10. The apparatus of claim 9, wherein the microposts are arranged such that microfiber strands received by the apparatus form a plurality of segments at least some of which are aligned substantially parallel to one another.

11. The apparatus of claim 9, wherein the microposts are configured to receive the microfiber strand to form a plurality of microfiber segments, wherein at least some of the microfibers are arranged in multiple layers stacked to form a three-dimensional construct.

12. The apparatus of claim 9, wherein the microfiber is a biomaterial selected from the group consisting of collagen, elastin, hyaluronic acid, fibrinogen, fibrin, fibronectin, silk, alginate, and pluronic.

13. The apparatus of claim 9, wherein the microposts are disposed in a reservoir configured to receive a fluid bath in which the scaffold may be formed.

14. The apparatus of claim 9, further including a 3D printing device configured to dispense a microfiber strand; and
wherein the reservoir and microposts are movable in translation relative to the 3D printing device.

15. The apparatus of claim 9, further including a 3D printing device configured to dispense a microfiber strand; and
wherein the reservoir and microposts are movable in rotation relative to the 3D printing device.

16. The apparatus of claim 9, further including a 3D printing device configured to dispense a microfiber strand;
the 3D printing device including a coaxial needle configured to dispense the microfiber strand in a hydrogel sheath.

17. The apparatus of claim 16, wherein the coaxial needle includes:
an inner conduit and an outer conduit;
the inner conduit defining an inner lumen configured to dispense the microfiber strand;
wherein an annular outer lumen is defined between the outer conduit and the inner conduit; the annular outer lumen being configured to dispense the hydrogel sheath; and
a flexible extension tube extending from a tip of the outer conduit.

18. A method of making a scaffold construct, comprising:
dispensing a microfiber strand in a biocompatible hydrogel sheath; and
winding the microfiber strand around a plurality of microposts to form a plurality of segments ensheathed by a biocompatible hydrogel and arranged in an organized configuration.

19. The method of claim 18, wherein winding the microfiber strand to form an organized configuration of segments includes arranging at least some of the segments in substantial alignment with one another.

20. The method of claim 19, wherein the winding of the microfiber strand around the plurality of microposts forms a plurality of substantially parallel segments.

21. The method of claim 19, wherein arranging at least some of the microfiber segments in substantial alignment with one another includes arranging at least some of the plurality of microfibers aligned in the same plane as one another.

22. The method of claim 19, wherein arranging at least some of the microfiber segments in substantial alignment with one another includes arranging at least some of the microfibers in multiple layers stacked to form a three-dimensional construct.

23. The method of claim 18, wherein the microfiber is a biomaterial selected from the group consisting of collagen, elastin, hyaluronic acid, fibrinogen, fibrin, fibronectin, silk, alginate, and pluronic.

24. The method of claim 23, wherein the biomaterial is collagen.

25. The method of claim 24, wherein winding the collagen microfiber strand around the microposts is performed in a reservoir containing a bath of crosslinking solution.

26. The method of claim 25, wherein the crosslinking solution is a thrombin solution.

27. The method of claim 24, wherein winding the collagen microfiber strand around the microposts is performed in a reservoir containing a bath of crosslinking solution.

28. The method of claim 27, wherein the crosslinking solution includes Factor XIII.

29. The method of claim 27, further including relocating the reservoir containing the bath of crosslinking solution and the microposts in translation and rotation to ensure a known orientation with respect to a 3D printing device.

30. The method of claim 18, wherein the biocompatible hydrogel is a cellular hydrogel; and
wherein the method further includes maintaining the microfiber and hydrogel scaffold under cell culture conditions for one or more days.

31. The method of claim 18, wherein winding the collagen microfiber strand around a plurality of microposts includes winding multiple layers of collagen microfiber strand around the plurality of microposts to form a three-dimensional construct.

32. A method of treating volumetric muscle loss (VML), comprising:
affixing, within a VML wound site, a scaffold construct according to claim 1.
